SEQUENCE LISTING (42 sequences)

SEQ ID NO: 1 - DNA, 40 bp, Artificial Sequence
  Synthethic construct
gcgcggtcccgatttggtgtaaaattccctcagccctaca

SEQ ID NO: 2 - DNA, 40 bp, Artificial Sequence
  synthetic construct
gcgcggtcccgatttggtgtaaaattccctcagccctacg

SEQ ID NO: 3 - DNA, 40 bp, Artificial Sequence
  synthetic construct
gccggtgtaggtggtgtagaccatttctctcgtgcttggc

SEQ ID NO: 4 - DNA, 40 bp, Artificial Sequence
  synthetic construct
ggcgatgtagcgtgtgaagacactcctgaatagctgcctg

SEQ ID NO: 5 - DNA, 40 bp, Artificial Sequence
  synthetic construct
cggtgtagaccctggagagggtgtgcatatgtccggttgc

SEQ ID NO: 6 - DNA, 40 bp, Artificial Sequence
  synthetic construct
gctgtggggccagtttcgatgtagaaatccgatatgtaac

SEQ ID NO: 7 - DNA, 40 bp, Artificial Sequence
  synthetic construct
agaccgatgtaggcccagaagtgggtgtccgcacaagtgc

SEQ ID NO: 8 - DNA, 40 bp, Artificial Sequence
  synthetic construct
gtcgatgtagatggtgcagaccagccttcgttgtagtcca

SEQ ID NO: 9 - DNA, 40 bp, Artificial Sequence
  synthetic construct
gccggtgtaggtcacaaaggtgatccgatctcccgtatgt

SEQ ID NO: 10 - DNA, 40 bp, Artificial Sequence
  synthetic construct
agacccgcggttcgtcgagctcttgcggcagggtccaaga

SEQ ID NO: 11 - DNA, 40 bp, Artificial Sequence
  synthetic construct
gcgcggccccgatttggtgtaaaattccctcagccctaca

SEQ ID NO: 12 - DNA, 40 bp, Artificial Sequence
  synthetic construct
gcggcacggcaaggctgttcgttgtttgttgtgacgcccg

SEQ ID NO: 13 - DNA, 40 bp, Artificial Sequence
  synthetic construct
tggctcggtgtagactcccgcggagattcagttccctgtt

SEQ ID NO: 14 - DNA, 40 bp, Artificial Sequence
  synthetic construct
gcgcggtcccgatttggcgtaaaattccctcagccctaca

SEQ ID NO: 15 - DNA, 40 bp, Artificial Sequence
  synthetic construct
ggcgatgtagcagccgcagggcttacttctatacacctcg

SEQ ID NO: 16 - protein, 13 aa, Artificial Sequence
  synthetic construct
ARSRFGVKFPQPY

SEQ ID NO: 17 - protein, 13 aa, Artificial Sequence
  synthetic construct
ARSRFGVKFPQPY

SEQ ID NO: 18 - protein, 13 aa, Artificial Sequence
  synthetic construct
AGVGGVDHFSRAW

SEQ ID NO: 19 - protein, 13 aa, Artificial Sequence
  synthetic construct
  MISC_FEATURE (11)..(11): Xaa can be any amino acid or absent
GDVACEDTPEXLP

SEQ ID NO: 20 - protein, 13 aa, Artificial Sequence
  synthetic construct
RCRPWRGCAYVRL

SEQ ID NO: 21 - protein, 13 aa, Artificial Sequence
  synthetic construct
  MISC_FEATURE (8)..(8): Xaa can be any amino acid
  misc_feature (13)..(13): Xaa can be any naturally occurring amino acid
AVGPVSMXKSDMX

SEQ ID NO: 22 - protein, 13 aa, Artificial Sequence
  synthetic construct
  MISC_FEATURE (4)..(4): Xaa can be any amino acid or absent
RPMXAQKWVSAQV

SEQ ID NO: 23 - protein, 13 aa, Artificial Sequence
  synthetic construct
  MISC_FEATURE (12)..(12): Xaa can be any amino acid or absent
VDVDGADQPSLXS

SEQ ID NO: 24 - protein, 13 aa, Artificial Sequence
  synthetic construct
AGVGHKGDPISRM

SEQ ID NO: 25 - protein, 13 aa, Artificial Sequence
  synthetic construct
RPAVRRALAAGSK

SEQ ID NO: 26 - protein, 13 aa, Artificial Sequence
  synthetic construct
ARPRFGVKFPQPY

SEQ ID NO: 27 - protein, 13 aa, Artificial Sequence
  synthetic construct
AARQGCSLFVVTP

SEQ ID NO: 28 - protein, 13 aa, Artificial Sequence
  synthetic construct
WLGVDSRGDSVPC

SEQ ID NO: 29 - protein, 13 aa, Artificial Sequence
  synthetic construct
ARSRFGVKFPQPY

SEQ ID NO: 30 - protein, 13 aa, Artificial Sequence
  synthetic construct
GDVAAAGLTSIHL

SEQ ID NO: 31 - DNA, 37 bp, Artificial Sequence
  synthetic construct
cggtcccgatttggtgtaaaattccctcagccctaca

SEQ ID NO: 32 - DNA, 34 bp, Artificial Sequence
  synthetic construct
tcccgatttggtgtaaaattccctcagccctaca

SEQ ID NO: 33 - DNA, 35 bp, Artificial Sequence
  synthetic construct
gcgcggtcccgatttggtgtaaaattccctcagcc

SEQ ID NO: 34 - DNA, 30 bp, Artificial Sequence
  synthetic construct
gcgcggtcccgatttggtgtaaaattccct

SEQ ID NO: 35 - DNA, 32 bp, Artificial Sequence
  synthetic construct
cggtcccgatttggtgtaaaattccctcagcc

SEQ ID NO: 36 - DNA, 40 bp, Artificial Sequence
  synthetic construct
ttgttagatactcgctctcatccgccgtacaactggccag

SEQ ID NO: 37 - DNA, 40 bp, Artificial Sequence
  synthetic construct
actaccgatgcggacttatccgcacgtaccgctattgtag

SEQ ID NO: 38 - protein, 87 aa, Artificial Sequence
  synthetic construct
VSEDLRSRIEVLKRKVIEKVQHIQLLQKNVRAQLVDMKRLEVDIDIKIRSCRGSCSRALAREVDLKDYEDQQKQLEQVIAKDLLPSR

SEQ ID NO: 39 - protein, 328 aa, Artificial Sequence
  synthetic construct
DNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 40 - protein, 324 aa, Artificial Sequence
  synthetic construct
KMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 41 - protein, 5 aa, Artificial Sequence
  synthetic construct
MHRPY

SEQ ID NO: 42 - protein, 4 aa, Artificial Sequence
  synthetic construct
GHRP

(19) United States
(12) Patent Application Publication
(10) Pub. No.: US 2023/0201357 A1
(43) Pub. Date: Jun. 29, 2023

(54) D-DIMER-SPECIFIC APTAMERS AND METHODS OF USE IN DIAGNOSTICS, THERAPEUTIC AND THERANOSTIC PURPOSES

(71) Applicant: APTAMER DIAGNOSTIC, INC., Friso, TX (US)

(72) Inventors: Mohamad Ammar AYASS, Frisco, TX (US); Natalya GRIKO, Lewisville, TX (US); Angelo LUBAG, Plana, TX (US); Lina ABI MOSLEH, Frisco, TX (US)

(21) Appl. No.: 16/762,519

(22) PCT Filed: Nov. 5, 2018
(86) PCT No.: PCT/US2018/059149

§ 371 Date: May 8, 2020

Related U.S. Application Data
(60) Provisional application No. 62/583,467, filed on Nov. 8, 2017.

(51) Int. Cl. A61K 47/54; C12N 15/115; G01N 33/86

(52) U.S. Cl. CPC A61K 47/549; C12N 15/115; G01N 33/86; C12N 2310/16; G01N 2800/226; G01N 2333/75

(57) ABSTRACT

Disclosed are novel D-dimer specific aptamers and methods of their use.

### CROSS REFERENCE TO RELATED APPLICATIONS

This application claims priority to and is a United States National Phase Patent Application of International Patent Application Number PCT/US2018/059149, filed Nov. 5, 2018, which claims the benefit of U.S. Provisional Application No. 62/583,467, filed on Nov. 8, 2017, applications which are incorporated by reference herein.

### I. BACKGROUND

Current D-dimer diagnostic test products are limited in terms of speed, stability, specificity, reliability, storage and longevity requirements and costs. The closest devices and/or methods are: (1) the antibody based tests for D-dimer—either ELISA or turbidimetric; and (2) the Latex-based qualitative and semi-quantitative D-dimer tests (visual evaluation of the agglutination pattern indicating the level of D-dimer in the sample). All of the existing diagnostic tests for detecting D-dimer employ a methodologies that are less stable, more expensive, and mostly not point of-care capable. What is needed is a new set of diagnostic tests that do not suffer from these deficiencies.

### II. SUMMARY

Herein is described a number of DNA/RNA sequences (aptamers) that selectively bind D-dimer and related Fibrin Degradation Products (FDPs) and are demonstrated to have use for numerous clinical and research diagnostic and therapeutic targeted technologies.

In one aspect, disclosed herein are isolated nucleic acids encoding an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30, or any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. For example, disclosed herein are isolated nucleic acid comprising sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. Also disclosed herein are isolated nucleic acids wherein the nucleic acid sequence is a truncation of SEQ ID NO: 1 (such as, for example, SEQ ID NO: 16, SEQ ID NO: 17, SEQ ID NO: 18, SEQ ID NO: 19, or SEQ ID NO: 20).

In one aspect, the disclosed nucleic acids can be modified to incorporate a detectable tag (such as, for example, a latex bead, magnetic bead, fluorescence label; fluorescent probe, chemiluminescent labels, radiolabels, and/or nanoparticle probe).

It is understood and herein contemplated that the disclosed nucleic acids can be aptamers that bind D-dimer. The D-dimer aptamers and its derivatives are demonstrated to have preferential binding to D-dimer in solution, whole blood, blood sera, and in blood plasma in the relevant physiological concentration range. Quantitative, semi-quantitative, and qualitative methods that may or may not require separate equipment have been shown to give test values in concordance with current protocols for D-dimer approved for clinical use. Thus, disclosed herein are methods of detecting D-dimer in a subject comprising obtaining a biological sample (such as for example, a blood sample including, but not limited to whole blood, blood sera, or blood plasma) from the subject and measuring the concentration of D-dimer in the subject using one or more of the aptamers of any preceding aspect.

As the presence of high concentrations of D-dimer has been associated with the presence of deep venous thrombosis (DVT), pulmonary embolism (PE), disseminated intravascular coagulation (DIC), and/or any inflammatory condition where the inflammation affects the vasculature, detection of D-dimer can be used for the detection of the presence of deep venous thrombosis (DVT), pulmonary embolism (PE), or disseminated intravascular coagulation (DIC) as well as any inflammatory condition where the inflammation affects the vasculature. Accordingly, disclosed herein are methods of detecting deep venous thrombosis (DVT), pulmonary embolism (PE), disseminated intravascular coagulation (DIC), and/or any inflammatory condition where the inflammation affects the vasculature comprising obtaining a biological sample (such as for example, a blood sample including, but not limited to whole blood, blood sera, or blood plasma) from the subject and measuring the concentration of D-dimer in the biological sample using one or more of the aptamers of any preceding aspect; wherein a high D-dimer concentration (i.e., above 500 ng/mL or for subject older than 50 years old above the subjects age in years×10 ng/L or ×0.56 nmol/L); wherein a high concentration of D-dimer (i.e., above 500 ng/mL or for subject older than 50 years old above the subjects age in years×10 ng/L or ×0.56 nmol/L) indicates that the subject has DVT, PE, or DIC. In one aspect, when the concentration of D-dimer is high, the method can further comprises treating the subject from whom the biological sample was obtained by the administration of an anticoagulant.

Also disclosed herein are methods of treating deep venous thrombosis (DVT), pulmonary embolism (PE), and/or disseminated intravascular coagulation (DIC) comprising administering to a subject an anticoagulant (such as, for example bivalirudin (ANGIOMAX®), antithrombin III, argatroban (ACOVA®), dabigatran (PRADAXA®), heparin, warfarin (COUMADIN®), apixaban (ELIQUIS®), edoxaban (SAVAYSA®), enoxaparin (LOVENOX®), fondaparinux (ARIXTRA®), and rivaroaxaban (XARELTO®)) conjugated to one or more of the aptamers of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows an illustration of the SELEX protocol for the development of a D-dimer specific aptamer. A library containing 10¹⁵ random sequences (40 nucleotides each) was pre-cleared against Br-CN sepharose beads (negative selection). The Flow through from the negative selection step was applied to D-dimer that is immobilized on Br-CN sepharose beads. After multiple washing steps the bound material is eluted and amplified using a PCR step and applied for a second round of selection. After six rounds of selection, the eluted material was counter selected by mixing it with D-dimer depleted serum proteins that are immobilized on Br-CN sepharose beads. The flow through was taken through two more rounds of positive selection and a library was prepared using the material eluted from the eighth round and subjected to next generations sequencing. Please refer to materials and methods for a detailed description of each step.

FIG. 2A shows a comparison of the relative enrichment of 15 Aptamers (ADI1701-ADI1715) over 9 rounds of selection. Counter selection using serum depleted on D-dimer was applied on step 7. Each line exemplifies the abundance of an aptamer through the different stages of selection as indicated by the number of reads. The Y-axis represents the number of reads for each aptamer whereas the X-axis represents the selection step. ADI1701 is the most abundant sequence in our pool of aptamers represented by the highest number of reads obtained from sequencing.

FIG. 2B shows the relative enrichment of ADI1701 Aptamer over 9 rounds of selection. The fold increase in the number of reads for ADI1701 is calculated relative to the number of reads acquired from the first round of selection. Counter selection using serum depleted from D-dimer was applied on step 7. There is over 180 fold enrichment in ADI1701 aptamer after the 9^{th} round of selection.

FIG. 3A shows the predicted Motifs and Motif Locations in the 40-mer aptamer sequences for Targeting D-Dimer.

FIG. 3B shows the folding isomers (foldamers) of the four top sequences in Aptamer ADI1701 motif—ADI1701, ADI1702, ADI1711 and ADI1714 with enrichments reads of 12288, 1588, 230 and 170 after 9 rounds of selection, respectively. The red colored nucleotide in the structures ADI1702, ADI1711 and ADI1714 represent substitutions in reference to ADI1701. The secondary structure isomers, with corresponding energies (in kcal/mol), were calculated at sodium levels of 1×PBS (137 mM NaCl) at a temperature of 10° C. using the DNA Folding Software.

FIG. 4 shows binding of ADI1701 to pure D-dimer as seen on Native Gel Electrophoresis. About 22 pmol of either pure D-dimer (4 ug) and Fibrinogen were incubated in the absence or presence of 20 fold excess of ADI1701 (440 pmol). After 1 h of incubation the sample were loaded on an 8% acrylaminde gel and stained with either SYBR gold (A) or Coomassie blue stain (B).

FIGS. 5A and 5B show an ELISA Based Aptamer Binding Assay to D-Dimer. FIG. 6A shows 25 nM of the indicated biotinylated aptamer was incubated with D-Dimer protein immobilized on a 96 well ELISA plate. Absorbance was measured after incubation with streptavidin horse radish peroxidase bound to the biotinylated aptamer in the presence of the substrate 3,3′,5,5′-Tetramethylbenzidine (TMB). 3′ Biotin labeled ADI1701 bind better to D-dimer than that 5′ labeled aptamer. FIG. 6B shows a competition assay to determine the specificity of binding of ADI1701 to D-Dimer in the presence of ADI1701 as well as incubation in the presence of three scrambled sequences of ADI1701.

FIG. 6 shows an ELISA Based Competition Assay for Binding of ADI1701 to D-Dimer in the Presence of Excess of Non-Biotin Labelled aptamer. The Indicated concentration of 3′ Bioitn-ADI1701 was incubated with D-Dimer protein immobilized on a 96 well ELISA plate in the absence or presence of 100-fold excess of non-biotinylated ADI1701. Absorbance was measured after incubation with streptavidin horse radish peroxidase bound to the biotinylated aptamer in the presence of the substrate 3,3′,5,5′-Tetramethylbenzidine (TMB). The binding affinity of ADI1701 to thrombin is estimated to be 10 nM.

FIG. 7 shows ELISA Based Binding Assay for ADI1701 to D-Dimer. 25 nM of either 3′ Bioitnilated ADI1701, Truncation 1, Truncation 2, Truncation 3, Truncation 4, Truncation 5, scrambled sequence 1, scrambled sequence 2 and scrambled sequence 3 was incubated with D-Dimer protein immobilized on a 96 well ELISA plate. As a control, 3′ Biotin-ADI1701 was incubated with fibrinogen or two plasma samples from donors with low D-Dimer levels (<200 ng/ml) immobilized on the plate. Absorbance was measured after incubation with streptavidin horse radish peroxidase bound to the biotinylated aptamer in the presence of the substrate 3,3′,5,5′-Tetramethylbenzidine (TMB). Full length ADI1701 Shows the best binding to D-Dimer. Deleting three or five bases from the 5′ end of ADI1701 decreased the binding to D-dimer by 33%. ADI1701 did not bind to Fibrinogen or plasma proteins proving that the aptamer is specific and there is no cross reactivity. The values shown are subtracted from the absorbance measured in the presence of albumin that was used as the blank.

FIG. 8 shows direct binding of ADI1701 to D-dimer. Interaction between ADI1701 and D-dimer was analyzed by a SPR device, Openplex SPRi system from Horiba Scientific. Various amounts of ADI1701 (5 fmol, 50 fmmol and 500 fmol) were immobilized on a gold sensor chip, and D-dimer (10 nM) was injected to the flow cell, and ScrubberGen Ver. 2.0g software was used to subtract the references. D-dimer bound to ADI1701 in a dose dependent manner and affinity was 2.5×10-9 (M). Nonspecific binding of D-dimer to ADI1701scram was subtracted at the respective concentrations.

FIG. 9 shows the absorbing species in the Latex-Aptamer or Latex-Antibody system. The latex particles by themselves have low to negligible absorbance because of its ˜20 nm diameter, which would be about 30 nm with the conjugated aptamers (between 2 to 6 per bead particle). Panel A illustrates the attachment of aptamers to the latex bead and the possible aggregation if the aptamer binds to two sites in the protein; Panel B illustrates the same as in (A) but with a larger particle (100 nm) which would have a higher absorbance at the visible range when the particles come together; and Panel C the antibody immobilized on the surface of the latex bead before and after aggregation. The yellow arrows indicate the estimated hydrodynamic radius changes because of effective diameter increase with binding and/or aggregation.

FIGS. 10A and 10B show photomicrographs of Latex-Aptamer Beads at Different Preparations and at Two Extreme Levels of D-Dimer Concentration. FIG. 10A shows Initial Coagulation-Aggregation Testing for Specificity. The well concentration of the proteins are (serial dilution from left to right, per 150 uL total well volume): 10 nM, 5 nM, 2.5 nM and 1.25 nM of D-Dimer; 10 nM, 5 nM, 2.5 nM and 1.25 nM of Fibrinogen; 750 nM, 375 nM, 188 nM and 94 nM of the BSA. (Ref: NB3, p 19-20, Aug. 29, 2017). 50 ul of aptamer conjugated to the latex beads were added to each well. FIG. 10B shows 100× magnification (zoomed in); oblique lighting for images taken from the wells containing high and low levels of D-dimer.

FIG. 11 shows a sample calibration curve for ADI1701-Latex complex in the presence of increasing pure D-dimer at different concentrations similar to FIG. 10A method. Absorbance was measured at 405 nm in this turbidimetric assay.

FIG. 12 shows clinical Test Results for Agglutination Assay Time vs D-Dimer levels. A point of care aptamer-based agglutination assay was developed using a drop of blood collected by capillary puncture from a finger prick. One drop (approximately 30 ul) of blood was mixed immediarely with one drop of a suspension of latex beads conjugated with ADI1701 aptamers. The time it took to observe the appearanvce of visible agglutination was noted. Agglutination time above is an average between at least two observers. All tests are done at room temperate. Agglutination results were compared to D-Dimer levels in venous blood that were measured using Hemosil D-Dimer assay from Instrumentation Laboratory.

FIG. 13 shows EDC (1-ethyl-3-(3-dimethylaminopropyl)carbodiimide hydrochloride) schemes for linking the —COOH functionalized latex beads with the —NH₂-terminated aptamer. The scheme 1 is a shorter synthetic step using a more reactive intermediate while scheme 2 has an extra step that uses the more stable sulfo-NHS ester.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular D-dimer aptamer (such as, for example, any of SEQ ID Nos 1-15) is disclosed and discussed and a number of modifications that can be made to a number of molecules including the D-dimer aptamer (such as, for example, any of SEQ ID Nos 1-15) are discussed, specifically contemplated is each and every combination and permutation of D-dimer aptamer (such as, for example, any of SEQ ID Nos 1-15) i and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

D-Dimer is a degradation product of the Fibrin protein that is produced during fibrinolysis. It is present at low levels in healthy individuals (typically less than 200 ng/ml) whereas increased levels evidence the presence of intravascular coagulation and thrombotic disease. The D-dimer test is routinely used in the first-line assessment of patients suspected to suffer from venous thromboembolism (VTE), which can present as either deep vein thrombosis (DVT) or pulmonary embolism (PE).

Prior to the present disclosure, commercially available clinical D-dimer tests were exclusively based on antibody technology. The ELISA method requires a primary antibody (Ab) designed to bind to the target protein, and a secondary antibody (to bind to the primary antibody) that usually carries a signal generator in the form of an enzymatic amplification platform (e.g., horseradish peroxidase) or a fluorescent label (e.g., small molecule dye or nanoparticle). Turbidimetric immunoassay methods that use only one antibody require immobilization on beads (e.g., latex beads) that agglutinate to varying extents that depends on the level of D-dimer in the blood sample (see References below). In both major platforms, the expense to produce an inherently delicate antibody is the heart of the diagnostic system. Ab-based assay systems have to be stored with stabilizers in solution at a certain temperature (between 2-8° C.) and have limited shelf-life. The Ab-based reagents is often the most prohibitive in the in vitro diagnostics cost breakdown.

To remedy the problems with previously existing D-dimer platforms, disclosed herein are, in one aspect, are nucleic acid (DNA/RNA) or peptide sequences (i.e., aptamers) that selectively bind D-dimer and related Fibrin Degradation Products (FDPs) and are demonstrated to have use for numerous clinical and research diagnostic and therapeutic targeted technologies. Aptamers are molecules that interact with a target molecule (such as, for example D-dimer), preferably in a specific way. Typically aptamers are small nucleic acids ranging from 15-50 bases in length (or peptides of 5-17 amino acids in length) that fold into defined secondary and tertiary structures, such as stem-loops or G-quartets. Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹°, or 10⁻¹². Aptamers can bind the target molecule with a very high degree of specificity. For example, aptamers have been isolated that have greater than a 10000-fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000-fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Accordingly, disclosed herein are isolated nucleic acids encoding an amino acid sequence as set forth in Table 2, such as, for example, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30. It is understood and herein contemplated that variations (substitutions, insertions, deletions, truncation) to the nucleic acid sequence encoding an of the amino acids set forth in Table 2, such as, for example, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30 can occur without losing the ability to bind to the target and may increase or decrease binding affinity to the D-dimer target in a beneficial way. Truncations can occur either on the 3′ or 5′ end of the nucleic acid encoding the disclosed peptide, but preferably occurs on the 5′ end. Similarly, nucleic acid and/or amino acid derivatives or variants such as those described herein can also increase or decrease the binding affinity in a beneficial way to achieve optimum utility of the D-dimer-specific aptamers. Thus, in one aspect, disclosed herein are isolated nucleic acids encoding an amino acid sequence as set forth in Table 2, such as, for example, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30 or any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. For example, the isolated nucleic acids encoding an amino acid as set forth in Table 2, such as, for example, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30 can comprise an isolated nucleic acid comprising sequence as set forth in Table 1, including, but not limited to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.

1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

2. Peptides Variants

As discussed herein there are numerous variants of the aptamers (i.e. SEQ ID Nos: 16-30) disclosed herein that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 7 and 8 and are referred to as conservative substitutions.

TABLE 7
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 8
Amino Acid Substitutions Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 8, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO:16 is set forth in SEQ ID NO: 1. It is understood that for this mutation all of the nucleic acid sequences that encode this particular derivative of any of SEQ ID NOS: 16-30 are also disclosed including any degenerate nucleic acid sequences that encodes the particular polypeptide set forth in SEQ ID NOs: 16-30.

It is understood that there are numerous amino acid and peptide analogs which can be incorporated into the disclosed compositions. For example, there are numerous D amino acids or amino acids which have a different functional substituent then the amino acids shown in Table 7 and Table 8. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—CH═CH— (cis and trans), —COCH₂—CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

3. Nucleic Acids

There are a variety of molecules disclosed herein that are nucleic acid based, including for example the nucleic acids set forth in SEQ ID Nos: 1-15, or any fragments thereof (such as, for example SEQ ID Nos: 31-35. The disclosed nucleic acids are made up of for example, nucleotides, nucleotide analogs, or nucleotide substitutes. Non-limiting examples of these and other molecules are discussed herein. It is understood that for example, when a nucleic acid is DNA, the DNA will typically be made up of Adenine (A), Cytosine (C), Thymine (T), and Guanine (G). Similarly, when a nucleic acid is RNA, the RNA will typically be made up of A, C, G, and uracil (U). Likewise, it is understood that if, for example, an antisense molecule is introduced can be advantageous that the antisense molecule be made up of nucleotide analogs that reduce the degradation of the antisense molecule in the cellular environment.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (.psi.), hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙ CH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

It is also understood in a nucleotide substitute that both the sugar and the phosphate moieties of the nucleotide can be replaced, by for example an amide type linkage (aminoethylglycine) (PNA).

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

As noted herein, the isolated nucleic acids can be modified to comprise an substitution, insertion, or deletion of one or more nucleotides in the disclosed nucleic acid aptamer sequences set forth in Table 1, such as, for example, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto. In one aspect, the truncation can comprise a deletion of 1, 2, 3, 4, 5, 6, 7, 8, or 9 nucleotides from the 3′ or 5′ end of the aptamer. As disclosed herein, the 5′ end is not as essential as the 3′-end of the nucleotide. Thus, disclosed herein are isolated nucleic acids wherein the nucleic acid sequence is a truncation at the 5′ end. In one aspect, disclosed herein are isolated nucleic acids wherein the nucleic acid sequence is a truncation of SEQ ID NO: 1, such as, for example, any of the truncation mutants set forth in Table 4, including, but not limited to SEQ ID NO: 16, SEQ ID NO: 17, SEQ ID NO: 18, SEQ ID NO: 19, or SEQ ID NO: 20.

The D-dimer-specific aptamers disclosed herein (such as for example, any of the nucleic acids encoding an amino acid as set forth in SEQ ID NOs: 16-30, or any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto, including any of the nucleic acids set forth in Table 1, such as, for example, any of SEQ ID NO:s: 1-15, or any fragment, derivative, or variant thereof comprising at least 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto can be used in diagnostics, therapeutic and theranostic purposes. The disclosed aptamers have advantages over prior technologies including: (1) integrity—more stable biological probe than antibodies—in terms of biochemical resistance to change in the normal range of temperature, pressure, and chemical/biochemical exposure; (2) lower to absent immunogenicity (especially important for therapeutic purposes); (3) much simpler to synthesize—generally requires fewer steps resulting to better synthetic efficiency and purity, (4) less expensive to produce; (5) longer shelf-life—amenable to longer-term storage because of inherent stability; (6) faster results (within a few minutes); and (7) some configurations do not require an instrument and, therefore, are amenable to point-of-care clinical applications.

Due to the application in detection of D-dimer and/or detection of a clinical indication implicated by the presence of D-dimer (such as, for example, venous thrombosis (DVT), pulmonary embolism (PE), and/or disseminated intravascular coagulation (DIC)), it is understood and herein contemplated that modification of the disclosed aptamers (including any nucleic acid encoding the peptides set for in SEQ ID Nos: 16-30, including, but not limited to any of the nucleic acids set forth in SEQ ID Nos: 1-15) to comprise a detectable tag such as, for example, a latex bead, magnetic bead, fluorescence labels; fluorescent probes, chemiluminescent labels, radiolabels, and/or nanoparticle probe.

As used herein, a label or tag can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (Di1C18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow SGF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; ; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin EBG; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1;YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

Additionally, the interaction of the aptamer with protein (i.e, D-dimer) can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

The D-dimer aptamers disclosed herein and their derivatives are demonstrated to have preferential binding to D-dimer in solution, whole blood, blood sera, and in blood plasma in the relevant physiological concentration range. Quantitative, semi-quantitative, and qualitative methods that may or may not require separate equipment have been shown to give test values in concordance with current protocols for D-dimer approved for clinical use. More specifically, the aptamers and their derivatives selectively bind to D-dimer and a related series of FDPs which are indicators of many disease states such as but not limited to deep-vein thrombosis, pulmonary embolism, and and/or disseminated intravascular coagulation. This makes the aptamers capable of being used to quantify the level of D-dimer and FDPs in the blood and other appropriate samples. The disclosed nucleic acids can also be used to probe D-dimer and FDPs in biological samples and in vivo settings—a property that can be extended to diagnostic and therapeutic applications. Quantitative, semi-quantitative, and qualitative methods that do or do not require separate equipment have been shown to give test values in concordance with current protocols for D-dimer approved for clinical use.

### Fibrinolysis

The degradation of fibrin clots is mainly the function of plasmin, a serine protease that circulates as the inactive proenzyme, plasminogen. Plasminogen binds to both fibrinogen and fibrin, thereby being incorporated into a clot as it is formed. The activated form of tissue Plasminogen Activator (tPA) cleaves plasminogen to plasmin which then digests the fibrin; the result is a series of soluble FDPs to which neither plasmin nor plasminogen can bind. D-dimer is a unique 180 kDa protein FDP produced from cross-linked sections of fibrin, and is a reliable marker that has been correlated with a number of conditions, including, but not limited to deep venous thrombosis (DVT), pulmonary embolism (PE), and/or disseminated intravascular coagulation (DIC). This makes the aptamers capable of being used to quantify the level of D-dimer and FDPs in the blood (including who blood, plasma, and sera) and other appropriate samples. Qualitative, semi-quantitative, and quantitative methods of determining the levels of D-dimers using these aptamers and their derivatives, including, but not limited to bead-based technologies with and without the use of spectrometers, which may be lab-based or point-of-care applicable assays. Thus, disclosed herein are methods of detecting D-dimer in a subject comprising obtaining a biologic sample (such as for example, a blood sample including, but not limited to whole blood, blood sera, or blood plasma) from the subject and measuring the concentration of D-dimer in the subject using one or more of the aptamers of any preceding aspect. Methods for detecting D-dimer using the disclosed aptamers include but are not limited to Qualitative, semi-quantitative, and quantitative serum turbidimetric assays, fluorescence quenching, ELISA, ELIspot, flowcytometry, electrophoretic blots, D-dimer pulldown assay using aptamer conjugated beads or fluorescently labeled aptamers, DNA agglutination assay, Surface plasmon resonance (SPR), and/or optical biosensing.

### Associated Diseases

Knowing the blood D-dimer levels is extremely important when DVT, PE, DIC, and/or any inflammatory condition where the inflammation affects the vasculature are suspected. Timely decisions regarding some operations or medications that can be compromised or complicated by any of these conditions could be assisted by knowing, together with other clinical tests and observations, the D-dimer levels as soon as possible. The standard, FDA approved (CLIA regulated) D-dimer tests are mostly antibody-based turbidimetric or ELISA techniques that require a laboratory equipment and a significant amount of time. Sending the blood sample to the lab and reporting the results to the physician often takes hours to days. Only a few clinical tests have been introduced that could do point-of-care D-dimer testing—all antibody based. The nucleic acid-based detection methods and/or diagnostic methods disclosed herein can substitute, supplant or exceed current D-dimer diagnostic test products in terms of speed, stability, specificity, reliability, storage and longevity requirements and costs. The disclosed nucleic acids can also be used to probe D-dimer and FDPs in biological samples and in vivo settings—a property that can be extended to diagnostic and/or therapeutic applications.

As the D-dimer has been correlated with the presence of DVT, PE, and DIC, detection of D-dimer can be used for the detection of the presence of DVT, PE, DIC, and/or any inflammatory condition where the inflammation affects the vasculature. Accordingly, disclosed herein are methods of detecting DVT, PE, DIC, and/or any inflammatory condition where the inflammation affects the vasculature comprising obtaining a biologic sample (such as for example, a blood sample including, but not limited to whole blood, blood sera, or blood plasma) from the subject and measuring the concentration of D-dimer in the biologic sample sample using one or more of the aptamers of any preceding aspect; wherein a high D-dimer concentration (i.e., above 500 ng/mL or for subject older than 50 years old above the subjects age in years x 10 ng/L or x 0.56 nmol/L); wherein a high concentration of D-dimer (i.e., above 500 ng/mL or for subject older than 50 years old above the subjects age in years x 10 ng/L or x 0.56 nmol/L) indicates that the subject has DVT, PE, or DIC. In one aspect, when the concentration of D-dimer is high, the method can further comprises treating the subject from whom the biologic sample was obtained by the administration of an anticoagulant.

Therapeutic applications involving the same DNA/RNA sequences are amenable for in vivo testing and could be formulated for therapeutics based on its targeting nature and unique sequence. The isolated nucleic acids aptamers disclosed herein (such as any (including any nucleic acid encoding the peptides set for in SEQ ID Nos: 16-30, including, but not limited to any of the nucleic acids set forth in SEQ ID Nos: 1-15, as well as, any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.) can be used to deliver chemical species or physiologically relevant payloads that are related to the Fibrin degradation products such as D-dimer(s). In one example, the aptamer is expected to hone in on areas where clotting is dominant and deliver anticoagulant species. Other coagulation factors could be delivered to the site where it is necessary, thereby lowering the required dosage because of site-specific action. Accordingly, disclosed herein are methods of treating deep venous thrombosis (DVT), pulmonary embolism (PE), disseminated intravascular coagulation (DIC), and/or any inflammatory condition where the inflammation affects the vasculature comprising administering to a subject an anticoagulant (such as, for example bivalirudin (ANGIOMAX®), antithrombin III, argatroban (ACOVA®), dabigatran (PRADAXA®), heparin, warfarin (COUMADIN®), apixaban (ELIQUIS®), edoxaban (SAVAYSA®), enoxaparin (LOVENOX®), fondaparinux (ARIXTRA®), and rivaroaxaban (XARELTO®)) conjugated to one or more of the aptamers disclosed herein (such as any (including any nucleic acid encoding the peptides set for in SEQ ID Nos: 16-30, including, but not limited to any of the nucleic acids set forth in SEQ ID Nos: 1-15, as well as, any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.).

4. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended. For example, disclosed is a kit for detecting the presence of D-dimer or deep venous thrombosis (DVT), pulmonary embolism (PE), and/or disseminated intravascular coagulation (DIC) comprising any nucleotide encoding the amino acids set forth in SEQ ID Nos: 16-30 and/or the oligonucleotides set forth in SEQ ID Nos: 1-15, as well as any fragment, derivative, or variant thereof comprising at least 84, 85, 86, 87, 87.5, 88, 89, 90, 91, 92, 93, 94, 95, 96, 97, 98, 99, 99.1, 99.2, 99.3, 99.4, 99.5, 99.6, 99.7, 99.8, 99.9% sequence identity thereto.

While various embodiments of the present disclosure have been described above, it should be understood that they have been presented by way of example only, and not of limitation. Likewise, the various diagrams may depict an example architectural or other configuration for the invention, which is provided to aid in understanding the features and functionality that can be included in the invention. The invention is not restricted to the illustrated example architectures or configurations, but the desired features can be implemented using a variety of alternative architectures and configurations.

Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate that various embodiments be implemented to perform the recited functionality in the same order unless the context dictates otherwise.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present invention should not be limited by any of the above-described exemplary embodiments.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

D-Dimer, a 180-kDa protein, is a fibrin degradation product (FDP) produced when clots in the vascular system are broken down in a chain of reactions by fibrinolysis. The process of fibrinolysis consists of two types: Primary fibrinolysis occurs naturally as the body clears clots once the underlying scarred tissue heal. Secondary fibrinolysis can be induced with medications or occur as the result of disease.

During blood coagulation, activated thrombin converts fibrinogen into fibrin, the result of which is fibrin monomers that polymerase to form a soluble gel of non-cross-linked fibrin. Activated Factor XIII then converts the soluble fibrin gel to cross-linked fibrin. The formation of the fibrin clot triggers the production of plasmin, a major clot-lysing enzyme that circulates as the inactive proenzyme, plasminogen. Plasminogen binds to both fibrinogen and fibrin, thereby being incorporated into a clot as it is formed. The activated form of tissue Plasminogen Activator (tPA) cleaves plasminogen to plasmin which then digests the fibrin; the result is a series of soluble FDPs to which neither plasmin nor plasminogen can bind. D-Dimer is a unique FDP produced from cross-linked sections of fibrin, a reliable marker that has been correlated with a number of conditions. High level of D-dimer is commonly used as a biomarker for deep venous thrombosis (DVT), pulmonary embolism (PE) and disseminated intravascular coagulation (DIC).

(1) Diseases Associated with High D-Dimer

Knowing the blood D-Dimer levels is extremely important when deep venous thrombosis (DVT), pulmonary embolism (PE), or disseminated intravascular coagulation (DIC) is suspected. Timely decisions regarding some operations or medications that can be compromised or complicated by any of these conditions can be assisted by knowing, together with other clinical tests and observations, the D-Dimer levels as soon as possible.

Elevated levels of D-dimer have also been reported in cases of pro-coagulant state such as surgery, trauma, sickle cell disease, liver disease, severe infection, sepsis, inflammation, malignancy and in the elderly. Increased levels of D-dimer are also seen during normal pregnancy, but very high levels are associated with complications.

(2) Current Technologies

The standard, FDA approved (CLIA regulated) D-Dimer tests are high complexity antibody-based turbidimetric or ELISA techniques that require laboratory equipment and a significant amount of time and qualified personnel to perform the test. Sending the blood sample to the laboratory and reporting the results to the physician often takes hours to days. Only a few clinical tests have been introduced that could do point-of-care D-Dimer testing—all antibody based. The ELISA method requires a primary antibody (Ab) designed to bind to the target protein, and a secondary antibody (to bind to the primary antibody) that usually carries a signal generator in the form of an enzymatic amplification platform (e.g., horseradish peroxidase) or a fluorescent label (e.g., small molecule dye or nanoparticle). Turbidimetric immunoassay methods that use only one antibody require immobilization on beads (e.g., latex beads) that agglutinate to varying extents that depends on the level of D-Dimer in the blood sample. In both major platforms (i.e. ELISA and turbidimetric), the expense to produce an inherently delicate antibody is the heart of the diagnostic system. Ab-based assay systems have to be stored with stabilizers in solution at certain temperature (typically 4° C.) and have limited shelf life. The Ab-based reagents are often the most prohibitive in the in vitro diagnostics cost breakdown.

(3) Aptamers

Aptamers are RNA or DNA oligonucleotides that, through their 3-dimensional structures, bind to specific target molecules with high affinity and specificity similar to antibodies. Aptamers have a lot of advantages over antibodies: they are synthetically created reducing the cost and time of production, there is no lot-to-lot variability, they are stable at room temperature, they are smaller than antibody proteins and can easily be modified chemically.

The target molecules of aptamers can be small molecules, large biomolecules, and even cells. Since their advent in 1990, aptamers have been developed for use in diagnostics. Aptamers specific to the D-Dimer series of FDPs have been published in a patent (US 20140296500 A1, Pub. date: Oct. 2, 2014). These patented DNA aptamers have had 4 years of exposure to the public. However, no useful clinical diagnostic test has come out of the sequences published. The nucleic selection process has become the shortest part of the aptamer research and development. However, the development of the testing platform and validation of the diagnostic protocols or device has always been the bottleneck for clinical diagnostics.

(4) Advantages

The inherent high affinity, specificity, biochemical stability, low to absent immunogenicity, fast production, and relatively low cost of production makes aptamers very desirable as substitutes for antibodies in diagnostics and future therapeutic applications. Aptamers are often referred to as “synthetic” antibodies.

(5) SELEX

A combinatorial process called SELEX (systematic evolution of ligands by exponential enrichment) has been developed in the Gold Laboratory (Univ. of Colorado, Boulder) in 1990. It is a systematic selection process that combines the power of biochemical selection with polymerase-chain reaction (PCR) through a series of binding-elution processes and nucleic acid amplifications. It amplifies the DNA or RNA after an elution cycle involving binding to immobilized targets. The repeated amplification of only the eluted target-binding sequences—initially from a pool of 10¹² to 10¹⁶ different nucleic acids after an elution step—allows for the selective amplification of the strongest binding nucleic acids.

On December 2004, the U.S. Food and Drug Administration (FDA) awarded OSI Pharmaceuticals the first aptamer-based drug for the treatment for age-related macular degeneration (AMD), called Macugen™. In addition, the company NeoVentures Biotechnology Inc. has successfully commercialized the first aptamer based diagnostic platform for analysis of mycotoxins in grain. Many contract companies develop aptamers and aptabodies to replace antibodies in research, diagnostic platforms, drug discovery, and therapeutics such as Aptagen LLC and Base Pair Biotechnologies.

At Aptamer Diagnostic, Inc. a DNA aptamer-specific for D-Dimer series of fibrin degradation products was identified, a critical blood clot level index. ADI1701 binds to D-dimer and FDPs with high specificity and a binding affinity in the nM range. Due to its binding properties, ADI1701 has the ability to replace the antibody-based technology for D-dimer detection in plasma samples, whether ELISA or agglutination based. D-dimer quantification from plasma from 20 donor samples shows correlation with the FDA approved test. A rapid agglutination assay was developed utilizing latex beads conjugated with a highly specific D-dimer aptamer ADI1701 that can be developed to a point-of-care-test. The Cross-linked Fibrin Degradation products present in a drop of plasma and whole blood bind to the aptamer-coated latex beads. This results in visible agglutination that occurs within a time cut-off of a few minutes when the concentration of D-dimer is above a set threshold (400 ng/ml). Aptamer discovery does not only lower the cost of D-Dimer testing but also introduces longer-lasting test kits for laboratories and point-of-care testing for medical care providers.

b) Results

(1) Selection of Aptamer Against Human D-Dimer:

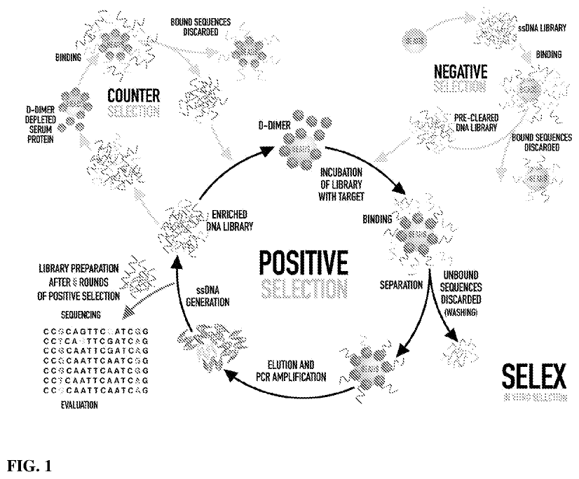

For the selection of a DNA aptamer against Human D-dimer, purified Human D-dimer protein was immobilized on CNBr-Activated sepharose beads using established protocol (see materials and methods). An aptamer pool consisting of 10¹⁵ random DNA sequences (Trilink) was pre-cleared against CNBr-Activated sepharose beads that are not crosslinked to D-dimer (FIG. 1). This step removes aptamers that non-specifically bind to the beads. The eluted material was collected and taken through the first round of positive selection (FIG. 1). The eluted library was incubated with Human D-dimer immobilized on CNBr-Activated sepharose beads and the aptamers that specifically bound to the D-dimer were eluted and amplified using a PCR Step. This process was repeated five times, using the amplified eluted material as input for the next round of positive selection. On step seven counter-selection was introduced against human serum depleted of D-dimer (refer to material and method section) that is immobilized on CNBr-Activated sepharose beads. Counter selection removes aptamers that show cross reactivity with other proteins that are available in serum. The unbound aptamers were taken through two final rounds of positive selection completing the SELEX process with a total of 9 rounds of selection.

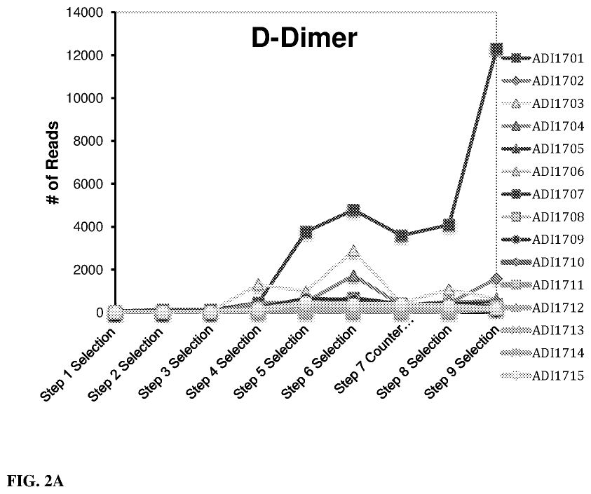

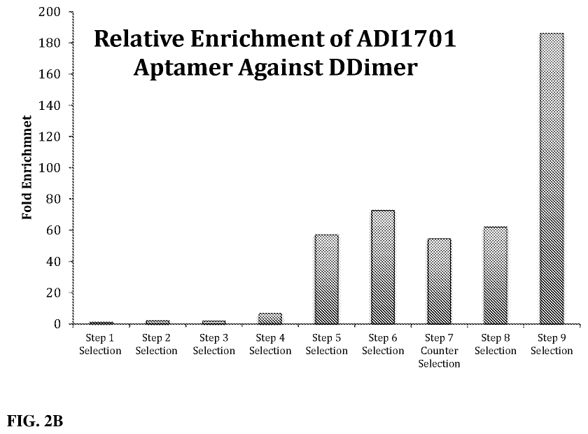

A fraction of the eluted material from every step of the SELEX process (eight positive and one counter selection) was subjected to next generation sequencing. FIG. 2A represents the relative enrichment of the top fifteen aptamers through each stage of the selection process. Aptamer ADI1701 is the most enriched sequence in the aptamer pool as it shows the highest number of reads starting on step 5 of positive selection (FIG. 2A). This is clearly illustrated in FIG. 2B that shows 60 fold increase of ADI1701 by step 5 of selection and an increases to over 180 folds by step 9 as compared to the first round. It is interesting to note that on step 7 of counter selection no significant decrease in ADI1701 was observed.

The 40-base long DNA sequences of the top 15-aptamer candidates targeting D-dimer were chosen from the starting 10¹⁵ oligonucleotides after 9 rounds of selection. The relative abundance of each aptamer is summarized in Table 1. It is important to note that the top two aptamers, ADI1701 and ADI1702 differ only by one base; ADI1701 has an adenine on position 40 whereas ADI1702 has a guanine. This provided the confidence in the specificity of binding of the ADI1701 sequence to D-dimer.

(2) Motif Analysis of Top 15 Aptamers:

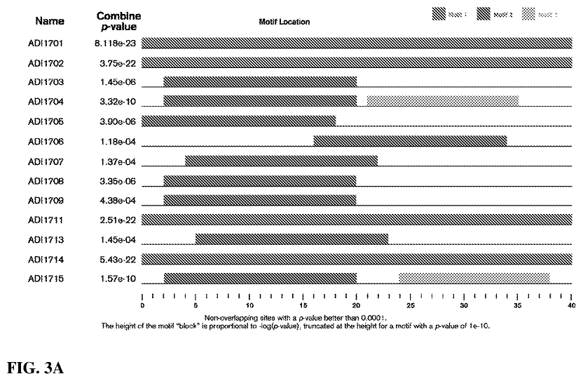

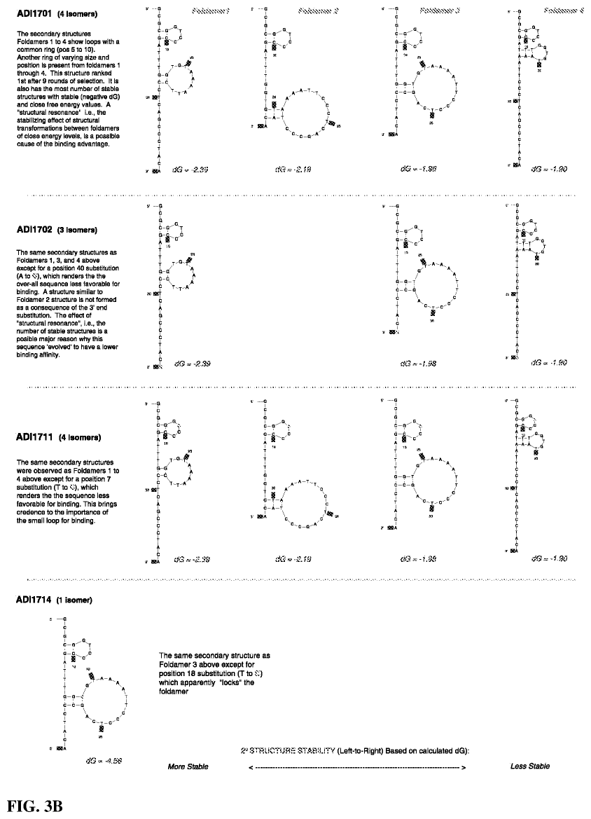

Motifs that are common between the top 15-aptamer sequences are shown in series of bases in fonts of red, blue and green (Table 1). MEME (version 4.12.0) from Galaxy was used to find the motifs in the set of 15 sequences (ADI1701 . . . ADI1715). MEME is a tool for performing de novo motif discovery in DNA, RNA, and proteins. MEME uses an algorithm called Expectation Maximization to find short patterns of nucleic acids or amino acids that occur more frequently in the input sequences that would be expected by chance. Because these patterns need not be exact matches, they are described using a matrix: the Position Specific Scoring Matrix (PSSM). Once MEME has generated the PSSM, it uses a second algorithm, MAST, to find the best matches to that PSSM in the input data. The p-value tells you how well a particular site matches the PSSM found by MEME. The smaller the p-value, the more significant the match. The number of motifs reported is limited to top 3 and each motif is in a different color. The discovered motifs have conserved regions, shown in FIG. 3A. The more conserved bases are thought to be involved intimately in the aptamer's molecular interactions with the D-dimer, either by direct binding or preserving the 3-D structure. Aptamers ADI1704 and ADI1715 have both blue and green motifs indicating that these two short motifs (18 and 14 bases, respectively) bind to different parts of the D-dimer.

TABLE 1

| | | | Number | SEQ |
|---|---|---|---|---|
| | Sequence | Rank | of Reads | ID No. |
| ADI1701 | GCGCGGTCCCGATTTGGTGTAAAATTCCCTCAGCCCTACA | 1 | 12288 | 1 |
| ADI1702 | GCGCGGTCCCGATTTGGTGTAAAATTCCCTCAGCCCTACG | 2 | 1588 | 2 |
| ADI1703 | GCCGGTGTAGGTGGTGTAGACCATTTCTCTCGTGCTTGGC | 3 | 643 | 3 |
| ADI1704 | GGCGATGTAGCGTGTGAAGACACTCCTGAATAGCTGCCTG | 5 | 530 | 4 |
| ADI1705 | CGGTGTAGACCCTGGAGAGGGTGTGCATATGTCCGGTTGC | 7 | 489 | 5 |
| ADI1706 | GCTGTGGGGCCAGTTTCGATGTAGAAATCCGATATGTAAC | 8 | 426 | 6 |
| ADI1707 | AGACCGATGTAGGCCCAGAAGTGGGTGTCCGCACAAGTGC | 9 | 386 | 7 |
| ADI1708 | GTCGATGTAGATGGTGCAGACCAGCCTTCGTTGTAGTCCA | 10 | 354 | 8 |
| ADI1709 | GCCGGTGTAGGTCACAAAGGTGATCCGATCTCCCGTATGT | 12 | 243 | 9 |
| ADI1710 | AGACCCGCGGTTCGTCGAGCTCTTGCGGCAGGGTCCAAGA | 13 | 230 | 10 |
| ADI1711 | GCGCGGCCCCGATTTGGTGTAAAATTCCCTCAGCCCTACA | 14 | 213 | 11 |
| ADI1712 | GCGGCACGGCAAGGCTGTTCGTTGTTTGTTGTGACGCCCG | 15 | 192 | 12 |
| ADI1713 | TGGCTCGGTGTAGACTCCCGCGGAGATTCAGTTCCCTGTT | 16 | 187 | 13 |
| ADI1714 | GCGCGGTCCCGATTTGGCGTAAAATTCCCTCAGCCCTACA | 17 | 170 | 14 |
| ADI1715 | GGCGATGTAGCAGCCGCAGGGCTTACTTCTATACACCTCG | 18 | 164 | 15 |

Nucleotide Sequence of the most abundant aptamers obtained after the 9^{th} round of
selection. The number of reads listed reflects the abundance of each sequence.
Highly abundant sequences have deeper coverage and therefore high number of
replicate reads. Three of the most highly abundant motifs identified in the
top fifteen highly abundant sequences in the D-dimer selection cluster pools
are shown above where the most abundant motif is shown in red, the second most
abundant is shown in blue and the third most abundant is shown in green.

In one aspect, it is understood and herein contemplated that the disclosed Aptamers can also be expressed in terms of the amino acids that they encode as set forth in Table 2.

TABLE 2
Amino Acid sequence of Aptamers

| Aptamer | | SEQ | | SEQ |
|---|---|---|---|---|
| ID: | Nucleic Acid Sequence | ID NO: | >Amino Acid Sequence | ID NO: |
| ADI1701 | GCGCGGTCCCGATTTGGTGTAAAATTCC | 1 | ARSRFGVKFPQPY | 16 |
| | CTCAGCCCTACA | | | |
| ADI1702 | GCGCGGTCCCGATTTGGTGTAAAATTCC | 2 | ARSRFGVKFPQPY | 17 |
| | CTCAGCCCTACG | | | |
| ADI1703 | GCCGGTGTAGGTGGTGTAGACCATTTCT | 3 | AGVGGVDHFSRAW | 18 |
| | CTCGTGCTTGGC | | | |
| ADI1704 | GGCGATGTAGCGTGTGAAGACACTCCTG | 4 | GDVACEDTPE*LP | 19 |
| | AATAGCTGCCTG | | | |
| ADI1705 | CGGTGTAGACCCTGGAGAGGGTGTGCAT | 5 | RCRPWRGCAYVRL | 20 |
| | ATGTCCGGTTGC | | | |
| ADI1706 | GCTGTGGGGCCAGTTTCGATGTAGAAAT | 6 | AVGPVSM*KSDM* | 21 |
| | CCGATATGTAAC | | | |
| ADI1707 | AGACCGATGTAGGCCCAGAAGTGGGTGT | 7 | RPM*AQKWWSAQV | 22 |
| | CCGCACAAGTGC | | | |
| ADI1708 | GTCGATGTAGATGGTGCAGACCAGCCTT | 8 | VDVDGADQPSL*S | 23 |
| | CGTTGTAGTCCA | | | |
| ADI1709 | GCCGGTGTAGGTCACAAAGGTGATCCGA | 9 | AGVGHKGDPISRM | 24 |
| | TCTCCCGTATGT | | | |
| ADI1710 | AGACCCGCGGTTCGTCGAGCTCTTGCGG | 10 | RPAVRRALAAGSK | 25 |
| | CAGGGTCCAAGA | | | |
| ADI1711 | GCGCGGCCCCGATTTGGTGTAAAATTCC | 11 | ARPRFGVKFPQPY | 26 |
| | CTCAGCCCTACA | | | |
| ADI1712 | GCGGCACGGCAAGGCTGTTCGTTGTTTG | 12 | AARQGCSLFVVTP | 27 |
| | TTGTGACGCCCG | | | |
| ADI1713 | TGGCTCGGTGTAGACTCCCGCGGAGATT | 13 | WLGVDSRGDSVPC | 28 |
| | CAGTTCCCTGTT | | | |
| ADI1714 | GCGCGGTCCCGATTTGGCGTAAAATTCC | 14 | ARSRFGVKFPQPY | 29 |
| | CTCAGCCCTACA | | | |
| ADI1715 | GGCGATGTAGCAGCCGCAGGGCTTACTT | 15 | GDVAAAGLTSIHL | 30 |
| | CTATACACCTCG | | | |

Two aptamers, both containing the 40-mer full red motifs, ranked first and second among the candidates—with aptamer one (ADI1701) having reads that about equal the next three ranked (aptamers ADI1702, ADI1703, and ADI1704) combined (Table 1). The 18-mer blue motif is almost always on the 5′ side of the 40-base length with the exception of aptamer ADI1706. The green motif has only been observed twice in the top 15 aptamers ranked.

The red motif surely binds better than the two others (blue and green)—indicating that it is most probable that all 40 of the nucleotides in the red motif are tightly involved in binding to D-dimer. This is evident from the fact that simply ADI1701 and ADI1702 are the top two enriched sequences and therefore the most abundant sequences that bound D-dimer. Both sequences are seen after the first round of selection (Table 3) and show an exponential increase in the number of reads and therefore abundance as the selection progresses, highly indicating that the sequences selected for are not a PCR artifact introduced during the SELEX process. The same sequence repeats again with ADI1711 and ADI1704 that have one base difference from ADI1701 (position 7 of ADI1711 is changed to cytosine from threonine and position 18 in ADI1714 is changed from a threonine to a cytosine). The latter sequences show less abundance through out the selection process, indicating weaker binding to D-dimer.

TABLE 3

| | Number of Reads at Selection Step | | | | | | | | |
|---|---|---|---|---|---|---|---|---|---|
| | 1 | 2 | 3 | 4 | 5 | 6 | 7 | 8 | 9 |
| ADI1701 | 66 | 129 | 117 | 450 | 3764 | 4789 | 3593 | 4089 | 12288 |
| ADI1702 | 1 | 18 | 9 | 29 | 333 | 298 | 327 | 421 | 1588 |
| ADI1711 | 1 | 5 | 0 | 3 | 37 | 38 | 36 | 45 | 213 |
| ADI1714 | 0 | 3 | 7 | 11 | 46 | 59 | 56 | 58 | 170 |

The number of reads for closely related aptamers (ADI1701, ADI1702, ADI1711 and ADI1714) at each selection step showed exponential enrichment for ADI1701 and ADI1702 starting with the first selection step.
ADI1711 and ADI1714 that differ from ADI1701 in one at positions 7 and 18 respectively do not show significant enrichments by the 9^{th} round of selection.

Based on their sequence, aptamers form specific structural regions induced by their sequence-dependent fold. The fact that DNA and RNA fragments fold to minimize energy into different possible configurations indicate that such aptamers be analyzed in that regard. The more different the aptamer candidates' foldamers are in terms of secondary (and therefore, 3D) structure and the greater their differences in corresponding energies, the more varied their binding affinities are expected. As such, the possibility of the isolation and study of the active, if not the most active, foldamer(s) must be ascertained. Structures that have very close energy levels can easily interchange in solution, therefore, the foldamers can be, in practical considerations, equivalent as they can interconvert without energy input or assistance (FIG. 3B). The sequences generated were each examined for stable secondary structures using online freely available programs—UNAfold and Mfold. The secondary structures in FIG. 3B represents the four most stable secondary structures for the D1s aptamer sequence. The next three aptamer candidates (designated ADI1702, ADI1711, and ADI1714) are predicted to have mostly the first two structures, shown below, in common. This indicates that the first two structures, being most stable (calculated dG=−2.36 and −2.18 kcal/mol) must be the dominant forms responsible for the D-dimer protein specificity.

The single base changes did not disrupt the predicted secondary structure of the aptamer (FIG. 3B) and explain why these candidates were in the top 15 enriched sequences. ADI1701, ADI1702 and ADI1711 are predicted to form the most stable foldamer1 with the lowest energy. The lowest energy conformation is often the major structure that is dominant. This property is taken advantaged of when a single stranded DNA is warmed up above its Tm and cooled down to low (˜4 C) temperature for a certain period of time. This ‘tempering’ or ‘annealing’ stage was also found to be necessary in the activation of the D-dimer aptamer. Substituting the last alanine in ADI1701 with guanine eliminates foldamer2 in ADI1702. ADI1714 has only one predicted secondary structure (foldamer3).

The 18-mer blue motif is almost always on the 5′ side of the 40-base length with the exception of aptamer ADI1706 (FIG. 3). The green motif has only been observed twice in the top 15 aptamers ranked. The blue motif found in ADI704, ADI1705, ADI1706, ADI1707, ADI1708, ADI1709, ADI1713, and ADI1715 vary by 4, 5, 9, 7, 3, 6, 9, and 7 bases, respectively, compared to the highest ranking aptamer candidate ADI1703 (at rank 3). The differences in reads are only in the hundreds indicating that the change in bases in the blue motif do not as much affect the affinity of binding as those affected by single base changes in aptamers with the red motif. The green motif is ranked very low and, at this point, considered the least significant since the bases in the 14-mer sequences are variable.

Based on the data obtained so far, ADI1701 aptamer was the foremost candidate based on the SELEX process and was chosen for analysis to determine the specificity of its binding to D-dimer (FIG. 2A).

(3) Secondary Structure Analysis of Aptamers:

Each sequences generated was examined for stable secondary structures using online freely available programs—UNAfold and Mfold (FIG. 3B). The secondary structures represent the four most stable structures for the ADI1701 aptamer sequence. The next 3-aptamer candidates (designated ADI1702, ADI1711, and ADI1714, see Table 1 and FIG. 3A) were simulated to have mostly the first (ADI1702) or the first and second structures (ADI1711 and ADI1714), shown in FIG. 3B, in common. This indicates that the first two structures, being most stable (calculated dG=−2.36 and −2.18 kcal/mol) must be the dominant forms responsible for the D-dimer protein specificity.

More studies such as x-ray crystallography, NMR, and CD spectroscopy must be done in order to ascertain the 3-D binding structure. Molecular modeling softwares for docking and structure optimization must be done concurrently to confirm these. The lowest energy conformation is often the dominant structure. This property takes advantage of when a single stranded DNA is warmed up above its Tm and cooled down to low temperature (˜4° C.) for a certain period of time. This ‘tempering’ or ‘annealing’ stage is necessary for the aptamer to acquire its tertiary structure and therefore bind to its target molecule.

(4) Specificity of Binding of ADI1701 to D-Dimer

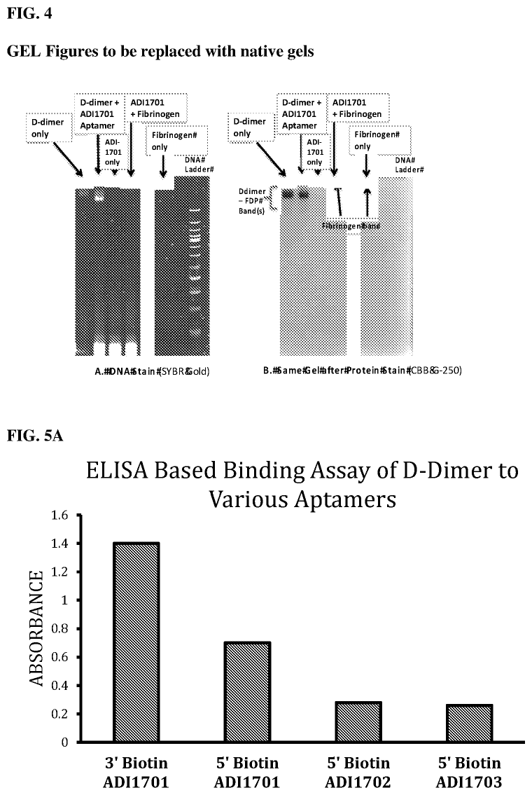

To test for the specificity of binding of ADI1701 to purified D-dimer, 20 fold excess of ADI1701 was incubated with 22 pmol of pure D-dimer (FIG. 4). The mixture was resolved on a native gel and stained with either SYBR gold to visualize the DNA aptamers or with protein stains Coomassie Brilliant Blue G-250 to stain the proteins. As seen in FIG. 4, ADI1701 (40 bases long) shows a clear shift from below the 100 base marker to above the 3 Kb marker when incubated with D-dimer. The DNA band corresponding to ADI1701 overlaps with the pure D-dimer protein as seen on the Coomassie stained gel. ADI1701 does not show any DNA band that overlaps with the protein corresponding to fibrinogen.

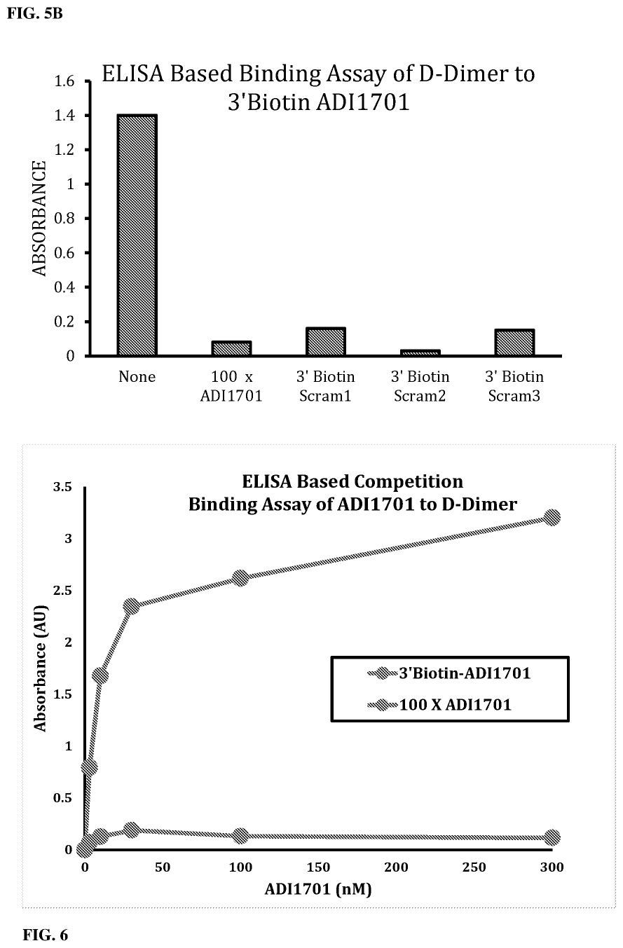

Binding of ADI1701 to D-Dimer was also tested in an ELISA based binding assay (FIGS. 5-7). Human D-Dimer protein immobilized on a 96 well ELISA plate (see material and methods) was incubated with ADI1701 with a biotin label on either the 3′ end (3′Biotin-ADI1701) or 5′ end (3′Biotin-ADI1701) (FIG. 5A). Absorbance was measured after incubation with streptavidin horseradish peroxidase bound to the biotinylated aptamer in the presence of the substrate 3,3′,5,5′-Tetramethylbensidine (TMB). 3′ labeled ADI1701 had higher binding to D-Dimer than the 5′ labeled aptamer. As expected, ADI1701 had higher binding to D-dimer than ADI1702 and ADI1703. Binding of 3′ Biotin-ADI1701 to D-dimer is completely diminished in the presence of 100-fold excess of non-biotinylated ADI1701 (FIG. 5B). None of the scrambled sequences (Scram1, Scram2 or Scram3) listed in table 4 showed any binding to D-dimer. Specificity of binding of ADI1701 to D-dimer was determined using the same ELISA assay by incubating the indicated concentration of ADI1701 to D-dimer immobilized on an ELISA plate in the absence or presence of 100-fold excess non-labelled ADI1701. FIG. 6 shows a saturation curve for binding of 5′Biotin-ADI1701 to D-Dimer. The binding was saturable and was diminished in the presence of excess unlabeled ADI1701. The estimated binding affinity of ADI1701 to D-dimer is in the order of 10 nM.

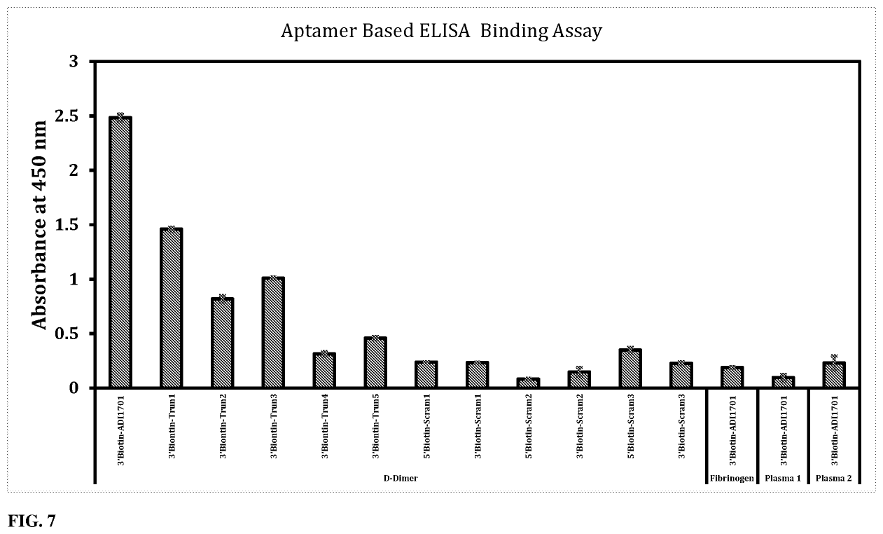

Various truncations were made to the 3′ Biotinylated-ADI1701 aptamer from the 5′ and 3′-ends. Deletions were determined based on the speculated foldamers presented in FIG. 3B. Deleting as little as 3 bases from the 5′-end or 5 bases from the 3′-end affected the binding of ADI1701 to D-dimer (FIG. 7). A list of all truncations and scrambled sequences are summarized in Table 4. Again, none of the scrambled sequences with Biotin label on either the 5′ or 3′-ends presented any binding to D-dimer. The binding of ADI1701 is specific to D-dimer since it failed to indicate any binding to either Fibrinogen or plasma proteins (with low D-dimer levels) that are immobilized on an ELISA plate (FIG. 7).

TABLE 4
List of truncations and scrambled sequences

| Sequence Name | Sequence | Description |
|---|---|---|
| ADI1701 | GCGCGGTCCCGATTTGGTGTAAAATTCCCT | Full Length |
| | CAGCCCTACA (SEQ ID NO: 1) | |
| ADI1701Trun1 | CGGTCCCGATTTGGTGTAAAATTCCCTCAG | Deleted first 3 bases |
| | CCCTACA-(SEQ ID NO: 31) | |
| ADI1701Trun2 | TCCCGATTTGGTGTAAAATTCCCTCAGCCC | Deleted first 6 bases |
| | TACA-(SEQ ID NO: 32) | |
| ADI1701Trun3 | GCGCGGTCCCGATTTGGTGTAAAATTCCCT | Deleted last 5 bases |
| | CAGCC-(SEQ ID NO: 33) | |
| ADI1701Trun4 | GCGCGGTCCCGATTTGGTGTAAAATTCCC | Deleted last 10 bases |
| | T-(SEQ ID NO: 34) | |
| ADI1701 Trun5 | CGGTCCCGATTTGGTGTAAAATTCCCTCAG | Deleted first 3 and last |
| | CC-(SEQ ID NO: 35) | 5 bases |
| Scrambled | TTGTTAGATACTCGCTCTCATCCGCCGTAC | |
| Sequence 1 | AACTGGCCAG-(SEQ ID NO: 36) | |
| Scrambled | ACTACCGATGCGGACTTATCCGCACGTACC | |
| Sequence 2 | GCTATTGTAG-(SEQ ID NO: 37) | |

List of truncations and scrambled sequences for ADI1701 that were tested in the
Elisa based binding assays.

(5) SPR Data

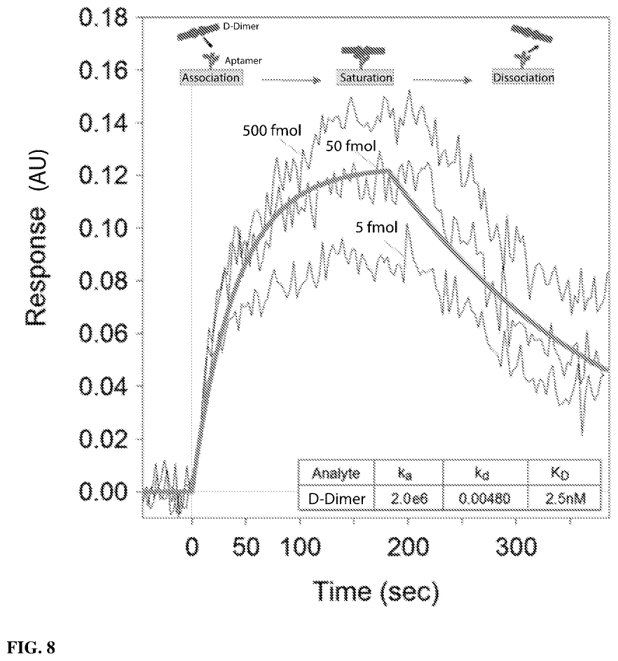

Further studies on the specificity and affinity of binding of ADI1701 to D-dimer were performed using Surface plasmon resonance (SPR). SPR has become an important optical bio-sensing technology in the areas of biochemistry, biology, and medical sciences because of its real-time, label-free, and noninvasive nature. Various amounts of biotinylated ADI1701 were immobilized on streptavidin coated gold surface and D-dimer to flow through. It is shown herein that ADI1701 binds directly to D-Dimer (FIG. 8). The specificity of binding was confirmed using a scrambled sequence of ADI1701 that disrupts the predicted motifs (FIG. 3A) and shows background binding to pure D-dimer. In ADI1701 binding to D-dimer, ka (1/ms) was 2.0×10⁶, kd (1/s) was 0.00480, and the KD value was 2.5 nM, respectively. Steady state affinity of D-dimer against ADI1701 was calculated to be 2.5 nM, approximately.

(6) Latex-Aptamer Studies

The latex bead technology has been utilized in many antibody-based assays in medical diagnostic testing. In the current D-dimer clinical assays, the latex beads typically 0.02 μm to 20 μm in diameter are coated with an antibody against anti-D-dimer. The beads agglutinate when they react with human plasma samples that contain D-dimer. Turbidimetry measures the loss of intensity in a transmitted light signal as it passes through a solution due to the scattering effect of particles suspended in it. The latex particles agglutination is proportional to the concentration of the D-dimer in the sample and can be measured by turbidimetry.

Using turbidimetric method, the same concept was applied using aptamers instead of antibodies. ADI1701 with an Amino functionalized group (NH₂—C₆-ADI1701) was conjugated to latex beads that are 20 μm in diameter with a Carboxylic acid modification using the 1-Ethyl-3-(3-dimethylaminopropyl)-carbodiimide (EDC) protocol (refer to materials and methods).

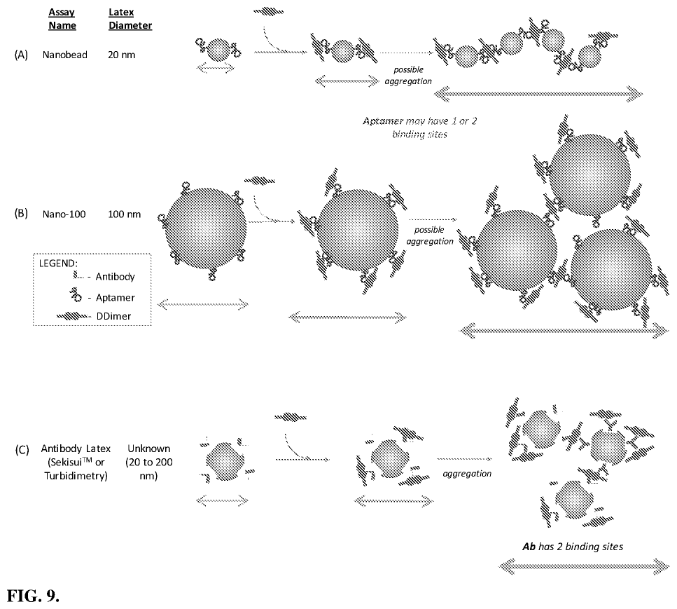

The synthesized ADI1701-latex bead suspension was exposed to between 1 to 2 minutes of 94° C. temperature and slowly cooled down by letting it stand to reach room temperature before refrigeration at 4° C. for storage. Freezing should be avoided. This step allows the greatest proportion of the active and most stable conformation of the aptamer to be achieved. Dilutions of pure D-dimer were incubated with ADI1701-latex complex for 10 minutes, after which absorbance was measured at 405 nm (FIG. 9). The fact that a D-dimer standard curve can be formed when the pure protein is used attests to the possibility of using the technique for homogeneous D-dimer testing, i.e., measuring the level in a blood sample without separating the D-dimer from the blood. FIG. 11 shows one example of such a curve. It is noted, though, that the same pure protein standard curve may not necessarily be used for turbidimetric measurements involving blood samples. Since the formation of the Latex-aptamer-D-dimer complex with a real blood sample includes major proteins, ions, and small molecules that affect its detection at a specific wavelength.

The signal enhancement observed during the D-dimer-Aptamer formation of larger particles by protein binding only (no aggregation) can come from the increased hydrodynamic radius. The capability of an aptamer to act like an antibody in the sense that it can bind two D-dimer molecules can contribute largely to the signal generated in aptamer based turbidimetric assays. The cartoon in FIG. 9 illustrates the theory for the aptamer (9A) and (9B) and the Ab-aggregation (9C) that is known to occur with Ab immobilized on beads.

(7) Development of Manual Assay—Qualitative to Semi-Quantitative

An antibody-based visual agglutination assay for D-dimer is commercially available form Sekisui™. It is intended for rapid qualitative evaluation of circulating derivatives of cross-linked fibrin degradation products (XL-FDP) in human plasma. A visual agglutination assay was developed utilizing latex beads coupled with ADI1701. The idea is for D-dimer and XLFDP present in plasma to bind to the coated latex beads, which results in visible agglutination occurring when the concentration is above the upper limit of detection for the assay. In order to achieve that, the dilution of the synthesized ADI1701-latex in the: (1) appropriate buffer, (2) the pH, and the (3) ionic composition/concentration are expected to change the ‘critical point’, i.e., the point at which visible aggregation of the aptamer-bead particles is facilitated by the concentration of the D-dimer. These three variables were investigated to determine this point at which the latex-aptamer mixture aggregates in the presence of certain levels of D-dimer in plasma for a start, due to the presence of potential interference factors in the blood.

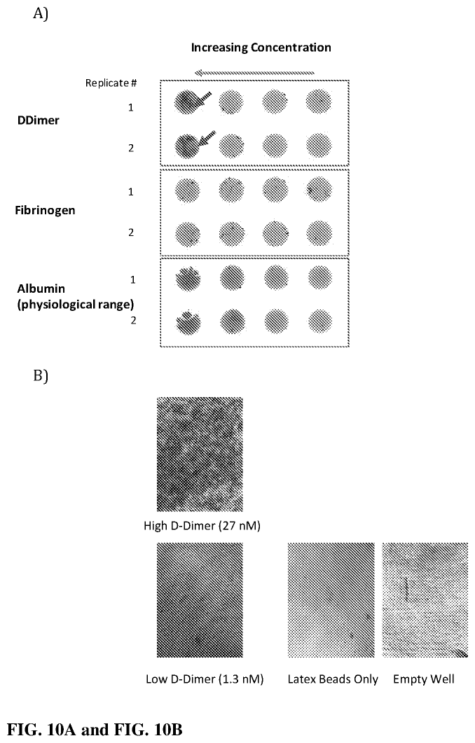

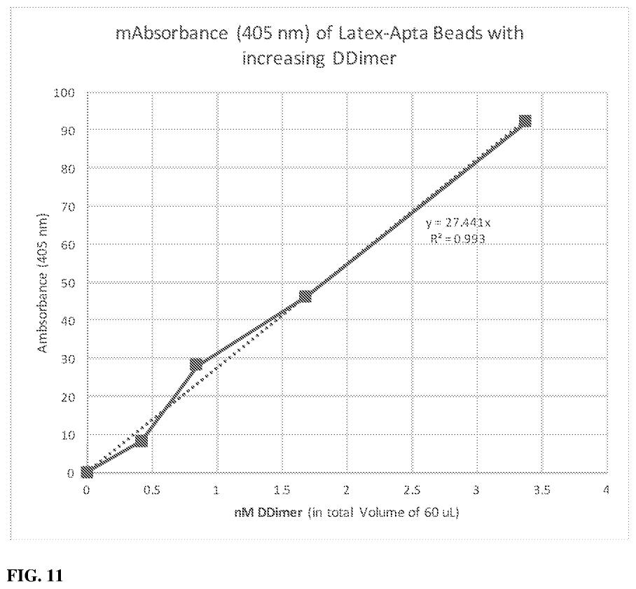

FIG. 10 represents an image of the wells section of a 96-well plate with the initial testing of coagulation. Agglutination is seen with D-dimer at concentrations that are lower than fibrinogen's (25 pmol for D-dimer as compared to 50 pmol of fibrinogen. The major protein constituent of plasma and serum is albumin. In order to check for interference, the agglutination of bovine serum albumin was tested around the physiological range with ADI1701-latex (FIG. 10). BSA did not show any agglutination even at the highest amount tested (18.8 nmol) which is above the physiologic range (physiologic range is 7.25 μmol/L as compared to 125 μmol/L tested at the highest concentration). 100×Magnification of the images taken shows clear agglutination of the ADI1701-latex beads when incubated with high levels of D-dimer (27 nM) but not in the presence of low D-dimer (1.3 nM) or the ADI1701-latex beads alone.

(8) Quantitative Turbidimetric Assay Development

The same 20 nm diameter latex beads conjugated to ADI1701 were adapted to develop the quantitative aptamer-latex assay system for turbidimetric method applied to human blood samples. As the illustration of the theory behind the quantitative turbidimetric assay (FIG. 9) indicates, the over-all signal change is brought about by the increased ‘turbidity’ is due to the immobilization of the D-dimer on the Latex-Aptamer surface that leads to larger particles and/or cross-linking of these particles. This binding increases the hydrodynamic ratio and the light absorption efficiency of the D-dimer species—purportedly due to the higher proportion of D-dimer in an immobilized/isotropic state (i.e., lower proportion of the protein in a continuously tumbling or ‘anisotropic’ state).

(9) Application of the Quantitative D-Dimer Assay to Blood Samples

The D-dimer concentrations for a blood sample can range from 50 to more than 3000 ng/mL. Levels lower than 200 ng/mL are often reported as <200 and are generally considered normal. A new analytical method that is calibrated for a range of 200-1000 ng/mL is, therefore, the bare minimum for a clinically relevant assay. A quantitative protocol validated for 50-5000 ng/mL D-dimer would be optimal.

The ideal range for a D-dimer assay translates to a standard curve concentration range of 0.278 nM (50 ng/ml) to 27.8 nM (5000 ng/ml) of the Fibrin Degradation Product (FDP) in the sample. The minimum range required mentioned above translates to 1.11 (˜200 ng/ml) to 5.55 nM (1000 ng/ml) D-dimer concentration in plasma. The assay has to be able to quantitatively delineate D-dimer levels in this range.

Plasma samples were the first blood samples examined because of their availability and stability—being frozen right after the centrifugation from whole blood. A comparison of the plasma D-dimer values calculated from the latex-aptamer assay versus the antibody-based turbidimetric results is shown in Table 5. The data represents the D-dimer levels in plasma from donors using the antibody based turbidimetric immunoassay method as compared to the aptamer based turbidimetruc based method. There is close concordance between the aptamer based and antibody based quantification of D-dimer levels in the plasma of donor samples with an average variation of 5% As mentioned above, the antibody based turbidimetric immunoassay method does not provide an absolute value for D-dimer levels that are less than 200 ng/ml.

TABLE 5
D-dimer quatification

D-Dimer Plasma Levels (ng/ml)

| Sample | Antibody Based | Aptamer Based |
|---|---|---|
| Number | Turbidimetry Assay | Turbidimetry Assay |
| 1 | 263 | 247 |
| 2 | <200 | 77 |
| 3 | 486 | 372 |
| 4 | <200 | 79 |
| 5 | 213 | 226 |
| 6 | <200 | 118 |

D-dimer quantification in plasma samples from donors using the aptamer-based versus antibody based turbidimetric method (using Hemosil D-Dimer assay from Instrumentation Laboratory)

(10) Application of the Qualitative D-Dimer Assay to Blood Samples

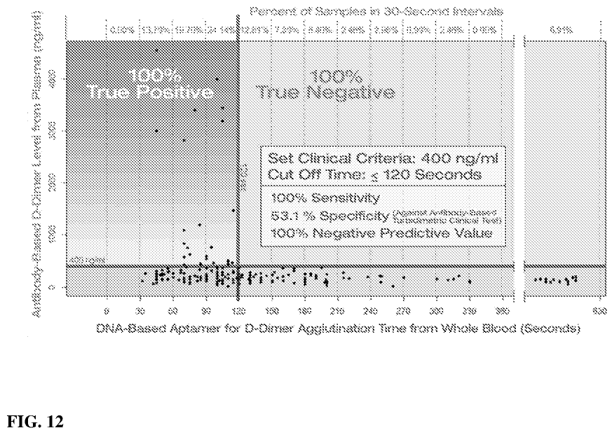

A qualitative assay was also developed that showed a visible agglutination when the D-dimer levels are above a certain threshold. FIG. 12 shows clinical Test Results for Agglutination Assay Time vs D-Dimer levels using a drop of blood collected by capillary puncture from a finger prick. One drop (approximately 30 ul) of blood was mixed immediately with one drop of a suspension of latex beads conjugated with ADI1701 aptamer. The time it took to observe the appearance of visible agglutination was noted. Agglutination results were compared to D-Dimer levels in venous blood that were measured using Hemosil D-Dimer assay from Instrumentation Laboratory. Results obtained from 105 donors show 100% sensitivity and 100% negative predictive value. Specificity of 53.1% was determined as compared to the Hemosil D-Dimer assay from Instrumentation Laboratory. The data obtained supports the claim that this aptamer series is suited for diagnostic applications including point of care use.

c) Discussion

Using the SELEX process an aptamer, ADI1701 was identified that binds to D-dimer and FDPs. ADI1701 was enriched after eight rounds of positive selection using pure immobilized human D-dimer, separated with one step of counter selection. The counter selection step was applied using plasma samples depleted of D-dimer in order to remove interfering factors that can bind to the aptamers non-specifically in a setting that mimics an actual patient sample. The studies disclosed herein show that ADI1701 bind to D-dimer with high specificity with a calculated binding affinity of 2.5 nM. Aptamers hold the ability to replace the antibody market due to the known properties that aptamers hold from cheaper production cost, scalability, no lot-to-lot variation, and higher shelf life stability. ADI1701 was used in the development of a qualitative as well as a quantitative assay for the detection and measurement of D-dimer in plasma samples from donor participants. The Aptamer-Latex D-Dimer (AD) Agglutination Method is a qualitative test developed to give a positive or negative determination based on visual evaluation based on agglutination of white particles in blood or plasma samples. The method was also advanced to be quantitative in nature and replace the antibody-based turbidimetric method that is currently available, based on the standard curve using pure D-dimer.

The adjustments of Later-Aptamer test solutions for this manual assay to optimize the ‘cut-off point’ for a specific level of D-dimer in the plasma was different from that of the ‘critical point’ for the quantitative turbidimetric assay. This assay was designed so that the aggregation of the ADI1701-latex—D-dimer interacting particles are strong enough to: (1) overcome the dispersive forces due to the solvating properties of water and ions in the buffer, and (2) form a chain or clump of particles big enough for the naked eye to observe (‘visual’ confirmation) when the D-dimer is at the target concentration, i.e., within the observation temporal window. It is important that the blood sample (plasma) is mixed thoroughly (about 6 to 10 figure eight stirring motions on the plate/mixing surface). The ‘critical points’, the levels of D-dimer (in ng/mL) at which the beads coagulate, are not exact values relative to antibody-based turbidimetric results. One important thing to note is that Ab-based results are almost exclusively measured from plasma, while the results in FIG. 10 are from the corresponding sera.

### 2. Example 2: Native PAGE

Native Gel studies were done on aptamer candidates against standards. It was found that, when pre-mixed before the run, a portion of the aptamer is bound with the D-dimer (and related series of Fibrin Degradation Products). This method qualitatively showed that the interaction seen in the native-PAGE was strong enough to merit the succeeding stages of the study.

From the Native Gel Studies, it can be seen that a bright band immediately below the D-dimer band indicates a portion of the aptamer with lagging electrophoretic mobility. This indicated a relatively strong interaction with the D-dimer protein. No such band was observed for Fibrinogen or BSA (in other runs. Ref Report Apr. 10-14, 2017; NB1-4-12-2017). SDS-PAGE verified, as expected, that the D-dimer standard used has many sub-units as expected.

### 3. Example 3: Immobilization of Aptamer on Latex Beads

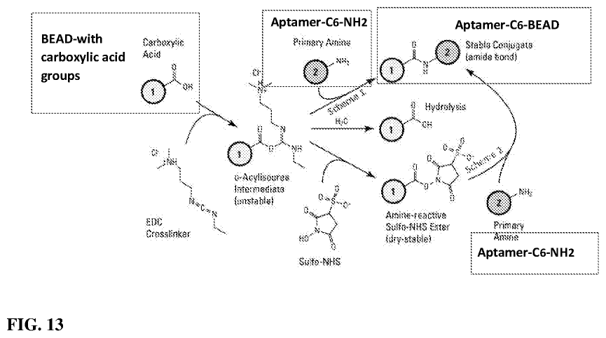

Carboxylic acid modified latex-beads ordered from ThermoScientific were conjugated with NH2-functionalized version of the D1s aptamer using the EDC protocol. The use of sulfo-NHS with the EDC reaction was also tested (FIG. 13). The NH₂—C₆-aptamer was conjugated on to the functionalized latex beads and cleanup was facilitated by dialysis (10 k MW cut-off) overnight in a suitable buffer. The synthesis and purifications details of the latex-aptamer beads were done using all available equipment including the characterization and quality control procedures (visual clarity, homogeneity, centrifugation, spectrophotometry, and DNA content check by Nanodrop™ and SYBR Gold™ staining after blotting on a sheet of nitrocellulose paper)

Seven separate synthesis trials (‘batch’) were done using the 20 nm beads. Since each batch has a slight variation from the previous one, comparisons were done amongst available batches when a new batch was synthesized to check which works better with the standards before applying it to the biological samples.

Dilution trials of the latex-aptamer was performed to find the concentration that can give a suitable calibration curve using the pure D-dimer protein. The fact that a D-dimer standard curve can be formed when the pure protein is used attests to the promising possibility of using the technique for homogeneous D-dimer testing, i.e., measuring the level in a blood sample without separating the D-dimer from the blood. The curve shown below is one example of such a curve obtained. It is noted, though, that the same pure protein standard curve can not necessarily be used for turbidimetric measurements involving blood samples. Since the formation of the Latex-aptamer-D-dimer complex with a real blood sample includes major proteins, ions, and small molecules that affect its detection at a specific wavelength, blood samples with verified D-dimer levels were used as standards during many tests in the development stages.

Dilution trials of the latex-aptamer was performed to find the concentration that can give a suitable calibration curve using the pure D-dimer protein. The fact that a D-dimer standard curve can be formed when the pure protein is used attests to the promising possibility of using the technique for homogeneous D-dimer testing, i.e., measuring the level in a blood sample without separating the D-dimer from the blood.

The coupling protocol using EDC (—COOH with —NH₂) used herein is described as follows.

a) General Two-Step Coupling Procedure:

Covalent Coupling of Oligonucleotides to Carboxylate-Modified Particles Via —COOH+—NH2 Binding. The first step is run at an acid pH to ensure that carboxylic acid groups are in COOH form. The second step is run at basic pH to ensure that amine groups are in NH2 form.

(1) Reagent Preparation

Nanosphere Beads comprising Carboxylate-modified polystyrene 4% solids are prepared in water. Water-Soluble Carbodiimide (WSC) Solution comprising no more than 2% (w/v) 1-ethyl-3-(3dimethylaminopropyl) carbodiimide (Sigma Chem. Co.) solution freshly prepared in deionized water. Other reagents include a pre-activation buffer comprising 0.05M KH2PO4, pH 4.5, a coupling buffer comprising 0.2M Borate Buffer, pH 8.5; an Oligonucleotide Solution: Calculated* volume of 100 uM nucleotide solution in Coupling (Buffer to calculated* final volume); a quenching Solution comprising 5 mM ethanolamine; and a Wash/Storage/Dialysis Buffer comprising PBS (pH 7.4) or 0.2 M Borate Buffer, pH 8.5 with 0.05% NaN3)

(2) Washing/Pre-Activation

To 1 ml microsphere suspension, add Pre-Activation Buffer. Place suspension on a magnet stirrer and maintain at room temperature (˜23° C.). Add WSC Solution (as calculated; may be diluted depending on starting amount of the nanosphere latex beads). NOTE: Add slowly at first attempt and stop as soon as coagulation or sudden cloudiness occurs. Note the volumes used (for next batch) Allow to react for 2 to 3 hours.

(3) Washing/Protein Coupling

Wash particle suspension in saline, and resuspend in 5 ml saline. Add equal volume of microsphere suspension to calculated equivalent* volume of Oligonucleotide Solution. Incubate at 22° C. for 20 hours (or at least overnight).

(4) Blocking/Washing

To neutralize surface carboxyl groups that are not bound to avidin, add Quenching Solution (Ethanolamine, 5 nM); may then add BSA (blocker) to a suitable concentration (no more than 2% w/v). Dialyse Latex-Aptamer suspension once overnight in Wash/Storage/dialysis Buffer at room temperature. Centrifuge at 15,000×g if needed (to remove coagulated spheres) or filter using 0.2 to 1 um filter, retaining the supernate.

### 4. Example 4: Development of Manual Assay—Qualitative to Semi-Quantitative

The dilution of the synthesized latex-aptamer in the: (1) appropriate buffer, (2) the pH, and the (3) ionic composition/concentration are expected to change the ‘critical point’, i.e., the point at which visible aggregation of the aptamer-bead particles is facilitated by the concentration of the D-dimer. These three variables were investigated to determine this point at which the latex-aptamer mixture aggregates in the presence of certain levels of D-dimer in serum. It can be seen that the D-dimer coagulated at a concentration that is lower than the fibrinogen's and much lower than that of BSA.

The adjustments of Latex-Aptamer test solutions for this manual assay to optimize the ‘cut-off point’ for a specific level of D-dimer in the blood was different from that of the ‘critical point’ for the quantitative turbidimetric assay below. This assay was designed so that the aggregation of the latex-Aptamer-D-dimer interacting particles are strong enough to: (1) overcome the dispersive forces due to the solvating properties of water and ions in the buffer, and (2) form a chain or clump of particles big enough for the naked eye to observe (‘visual’ confirmation) when the D-dimer is at the target concentration, i.e., within the observation temporal window. It is important that the blood sample (plasma or serum, as the case may be) is mixed thoroughly (about 6 to 10 figure eight stirring motions on the plate/mixing surface). One important thing to note is that Ab-based results are almost exclusively measured from plasma, while the results in latex-aptamer coagulation are from the corresponding sera.

The current delineation of the aggregation point is not as sharp and straightforward as desired using the 20 nm latex beads. It was decided to test 100 nm beads in future assays so that more of the aptamer can be immobilized on the surface of each particle. Another advantage of using a larger particle is that the aggregated Latex-Aptamer-D-dimer is bigger, which makes it amenable for absorbance at higher wavelengths after binding D-dimer. The synthesis and quality control of the first batch of the larger particle latex-aptamer (also from ThermoFisher™ and using the same EDC chemistry) is ongoing as of the writing of this report.

### 5. Example 5: Quantitative Turbidimetric Assay Development

The same 20 nm diameter latex beads conjugated to the aptamer as described in Section 3 was adapted to develop the quantitative latex-aptamer assay system for turbidimetric method applied to human blood samples. The over-all signal change is brought about by the increased ‘turbidity’ and is due to the immobilization of the D-dimer on the Latex-Aptamer surface that leads to larger particles and/or cross-linking of these particles. This binding increases the hydrodynamic ratio and the light absorption efficiency of the D-dimer species—purportedly due to the higher proportion of D-dimer in an immobilized/isotropic state (i.e., lower proportion of the protein in a continuously tumbling or ‘anisotropic’ state).

### 6. Example 6: Conclusions and Recommendations

DNA aptamers that bind specifically to D-dimer and related Fibrin Degradation Products (FDP) were selected using the SELEX protocols. The top aptamers were examined by electrophoresis, ELISA and SPRI based binding assays. Analogs of the top aptamer, designated as ADI1701 was found to be specific to the D-dimer series of FDPs by testing selected high-scoring sequences against pure D-dimer, fibrinogen, bovine serum albumin, and sera with known D-dimer levels.

The analogs of ADI1701 aptamer were prepared: (1) biotinylated (at both 5′- and 3′-positions); (2) truncated (at both the 5′- and 3′-positions); (3) had the sequence scrambled; and (4) latex bead-conjugated. The derivatives were tested for their feasibility to interrogate D-dimer in solution and in biological samples—blood plasma and sera. The combined results of the numerous tests with biotinylated analogs aptamers indicate that the 3′-derivatization confers greater binding affinity to the D-dimer compared to the 5′-conjugation. Initial estimates of Kd by SPR was circa 2.5 nM using the 3′-biotinyated ADI1701 aptamer.

The fluorescent dye-conjugated 5′-AlexaFluor488-D1s aptamer was determined to be a potential commercial quantitative aptamer derivative for homogeneous D-dimer assays. It showed sample D-dimer values that are in good concordance with the Ab-based turbidimetric (clinically approved) results within the clinically relevant range of 200 to 1000 ng/mL D-dimer levels in blood. The change in signal intensity as the D-dimer level increase in the homogeneous test mixture (no separation required) is that of fluorescence quenching, i.e., the fluorescence intensity at the 488 nm wavelength of emission of the AlexaFluor adduct decreases as the D-dimer increases in the relevant concentration range.

The Latex-Aptamer bead platform based on the ADI1701 sequence was developed, synthesized, and tested on human blood samples. Comparison of results using the Latex-Aptamer beads system showed potential for manual semi-quantitative and visible qualitative testing of D-dimer without the use of instruments, therefore, point-of-care suitable. The same Latex-Aptamer showed quantitative turbidimetric results that are in good concordance with antibody-based turbidimetric D-dimer levels in dozens of blood samples tested—especially using freshly isolated plasma from whole blood that was collected no more than 72 hours before plasma isolation.

The novel D-dimer-targeted Latex-Aptamer can still be improved for final commercial applications with extended testing and development. The use of larger beads and fine-tuning the aptamer loading and sequence evolution are expected to further modulate and improve the sensitivity, affinity, and dynamic range of the D-dimer quantification. The examination of binding nuances, addition of stabilizers (e.g., fibrin degradation products) especially in the preparation standards, and testing the limits of interfering species must be done before commercialization.

### 7. Example 7: D-Dimer Aptamer Selection Protocol

The following protocol was used in the SELEXbased screening for the D-dimer Aptamers: D-Dimer

a) 1. Immobilization of the Target Protein on Br-CN Activated Sepharose.

D-Dimer (MW 180 kDa) was immobilized on Br-CN activated sepharose at final concentration 1.5 mg of protein per 1 ml sepharose. Binding of D-Dimer was quantitated and confirmed using Pirce BCA protein Assay kit (cat #23227)

b) 2. Aptamers Selection

1. Dilute 10 nmole Aptamer library (1 tube TriLink biotechnologies) in 100 ul of DNase and RNase free water. Save 1 ul of the library for PCR (original library)
2. Add 100 ul of binding buffer (50 mM Tris pH 7.5, 150 mM NaCl, and 1 mM EDTA) to the diluted library and heat it at Hybex at 950 C for 10 min.
3. Cool down the library on ice for 10 min
4. Dilute the ssDNA library in 1 ml total of binding buffer. Save 5 ul for PCR (loading library)
5. Wash 100 ul of the sepharose beads (bed volume) alone, or the D-Dimer beads with 1 ml of the binding buffer 3 times (5 min. rotation between each wash)
6. Apply diluted library to the sepharose beads (negative selection), incubate for 30 min. at room temperature and collect the flow throw. Save 5 ul for PCR.
7. Apply the flow throw after negative selection to D-Dimer sepharose beads and incubate for 2 hours at room temperature.
8. Collect flow throw, save 5 ul for PCR.
9. Wash the beads 3 times with the binding buffer and collect the washes for PCR.
10. Elute the DNA library from the d-dimer beads:
i. Add 30 μmole of NaOH (0.2 ml of 0.15M stock), gently vortex and rotate at RT for 10 mins to elute the ssDNA from D-dimer
ii. The tube is spun down and the unretained ssDNA is added into a tube containing 30 μmole of acetic acid (0.2 ml of 0.15M stock) to neutralize the base.
iii. Buffer the solution by adding 40 μl of 3 M sodium acetate followed by 1 ml of cold 100% ETOH to precipitate the ssDNA.
iv. Repeat the NaOH elution process on the beads and precipitate the eluted ssDNA
v. Place the two tubes in −20 c for 2 h or overnight
vi. Spin at 13,200 rpm for 45 mins at 40 C
vii. Wash with 1 ml of 70% ETOH and
viii. Spin for 20 mins at 13,200 rpm at 40 C (repeat the wash 2×)
ix. Remove ETOH and air dry (10 min) or speed vacuum dry
x. Reconstitute each tube in 15 μl of water, incubate at 370 C on Hybex for 10 min, vortex and spin down.

c) PCR Amplification

PureTaq ready to go PCR Beads are lyophilized and need to be hydrated in a total volume of 25 μl. Add 5 μl of each of the forward and reverse primers supplied by Trilink (10 μM stock), 1 μl or 5 ul of the saved samples and H₂O up to 25 μl. Set up the following PCR program:
1. 95c for 5 mins
2. 95 c for 30 sec
3. 50 c for 30 sec
4. 72c for 30 sec
5. repeat steps 2-4 25 times
6. 72 c for 5 mins
7. hold at 4 c

Analyze PCR product using Bioanalazer. Average size of the aptamers is 80 nucleotides. PCR product of elution was collected. The combined PCR product is used for the second round of selection.

### 8. Example 8: Small-Fragment DNA/RNA—Aptamer Structures and Separation

Secondary Structure Variations in Aptamer Folding just like any polymer, the folding(s) of DNA and RNA are dictated by their sequence and environmental conditions. The permutations of bond-rotations and interactions increase exponentially as the oligonucleotide sequence length increases. The simulation of the candidate aptamers' secondary structure folding is, therefore, mandatory to help explain and (simulate) tertiary structures and concomitant binding—specificities, affinities and all.

Examination of Folding Isomers (“Foldamer”) Rationale: The fact that DNA and RNA fragments fold to minimize energy into different possible configurations indicate that such aptamers be analyzed in that regard. The more different the aptamer candidates' foldamers are in terms of secondary (and therefore, 3D) structure and the greater their differences in corresponding energies, the more varied their binding affinities are expected. As such, the possibility of the isolation and study of the active, if not the most active, foldamer(s) must be ascertained (if at all necessary). Structures that have very close energy levels can easily interchange in solution, therefore, the foldamers can be, in practical considerations, equivalent as they can interconvert without energy input or assistance.

Only a few techniques are known to be able to resolve DNA sequences with one base pair difference (deletion or substitution). Even more elusive to find and validate is a technique that can resolve a specific secondary structure of the same DNA sequence. Varying attempts have been made to solve such a problem. Besides Nuclear magnetic Resonance, only two techniques are published to have been used successfully to study phenomena in solution and only two that involve and/or close to achieving foldamer separations: electrophoresis and HPLC.

### 9. Example 9: D-dimer Concentrations of the Sera—Analytical Parameters

a) Concentration Range in Plasma

The D-dimer concentrations for a blood sample can range from 50 to more than 3000 ng/mL. Levels lower than 200 ng/mL are often reported as <200 and are generally considered normal. A new analytical method that is calibrated for a range of 200-1000 ng/mL is, therefore, the bare minimum for a clinically relevant assay. A quantitative protocol validated for 50-5000 ng/mL D-dimer would be optimal.

The ideal range for a D-dimer assay translates to a standard curve concentration range of 0.278 nM to 27.8 nM of the Fibrin Degradation Product (FDP) in the sample. The minimum range required mentioned above translates to 1.11 to 5.55 nM D-dimer concentration in plasma. The assay has to be able to quantitatively delineate D-dimer levels in this range.

For a constant Kd, a larger amount of D-dimer per volume of the reaction mixture would necessitate a lower amount of Aptamer sensor for the same number of D-dimer-Aptamer binding species to form. For a sample in a well with a low D-dimer concentration, an excess of aptamers will be necessary to enable the quantitation, i.e., for the D-dimer-Aptamer binding species detectable to correspond to the concentration of D-dimers.

b) Quantitative Estimates of Analytical Conditions Based on Kd and D-Dimer Levels in Plasma

For instance, for an Aptamer with a Kd of 700 nM, the following can be interpolated, when by definition 50% of the limiting reactant (Aptamer or D-dimer) is bound,

Kd=[Aptamer][D-dimer]/[Aptamer-D-dimer],

700 nM≈[(700 nM−0.01 nM)×(0.02 nM−0.01 nM)]/0.01 nM

which means that employing 700 nM Aptamer total concentration (which is much higher than the 0.01 nM, [Aptamer-D-dimer], a 0.02 nM D-dimer has to be initially in the sample if it is assumed that the 0.01 nM Aptamer-D-dimer product is readily detectable.

If [Aptamer]=700 nM,

Kd=700 nM≈[(700 nM−X nM)×(Y−X) nM)]/X nM

Which means that if a 700 nM Aptamer probe is used with a Kd=700 nM, Y, which is the original D-dimer content of the mixture (before binding), and X nM D-dimer is the final concentration present in the sample, an X nM Aptamer-D-dimer has to be in the upper range of the detection capability of the assay. In this example, the X nM concentration has to equal to Y/2.

If [Aptamer]=100 nM,

Kd=700 nM≈[(100 nM−X nM)×(Y−X) nM)]/X nM

In a case where 100 nM aptamer ( 1/7th of the 700 nM Kd) probe is used instead, the plasma D-dimer level, Y has to be transformed to X by the same fraction ( 1/7). In this case X= 1/7Y. The X nM or [Aptamer-D-dimer] has to be detectable in the assay. In this example, the X nM concentration also has to be <<the 100 nM aptamer probe.

c) Sample Mixture Calculation:

In a total assay mixture volume of 115 uL, a 5 uL plasma sample with 0.0139 pmol D-dimer (equivalent to 500 ng/mL=2.78 nM), the initial concentration of the D-dimer is 0.12 nM. In the steady-state mix with 132 nM available aptamer, it is calculated that 16% of the D-dimer (0.0022 μmol) is bound to the aptamer. For the 500 ng/mL plasma sample, it is therefore required that the equivalent 0.44 nM Aptamer-D-dimer complex be quantifiable (or 0.176 to 0.88 nM of the complex to be detectable, which corresponds to a 200-1000 ng/mL plasma D-dimer range). For one assay well with a total volume of 115 uL, this range represents a linear range of 2.64-13.2 pg of D-dimer.

### D. References

Bailey, T. L. and C. Elkan, Fitting a mixture model by expectation maximization to discover motifs in biopolymers. Proc Int Conf Intell Syst Mol Biol, 1994. 2: p. 28-36.
Cheung, K. K.-K. V., Antibody-based diagnostic and therapeutic innovations for human cancer. Comprehensive Therapy, 2000. 27(3): p. 183-194.
Daly, S. M., Biophotonics for blood analysis. 2015. 243-299.
Ellington, A. D. and J. W. Szostak, In vitro selection of RNA molecules that bind specific ligands. Nature, 1990. 346(6287): p. 818-22.
Gaffney, P. J., D-dimer History of Discovery, Characterization and Utility of this and other Fibrin Fragments. Fibrinolysis 1993. 7: p. 2-8.
Goff, K. A. B. K. K. P. G. N. F. D. A., Review of Rapid Diagnostic Tests Used by Antimicrobial Stewardship Programs Clinical Infectious Diseases, 15 Oct. 2014. 59(suppl 3): p. S134-S145.
Greenberg, C. S., D. V. Devine, and K. M. McCrae, Measurement of plasma fibrin D-dimer levels with the use of a monoclonal antibody coupled to latex beads. Am J Clin Pathol, 1987. 87(1): p. 94-100.
Gunn, G. R., 3rd, et al., From the bench to clinical practice: understanding the challenges and uncertainties in immunogenicity testing for biopharmaceuticals. Clin Exp Immunol, 2016. 184(2): p. 137-46.
Haven, M. C., G. A. Tetrault, and J. R. Schenken, Laboratory Instrumentation. 1994: Wiley.
Hunt, F. A., et al., Serum crosslinked fibrin (XDP) and fibrinogen/fibrin degradation products (FDP) in disorders associated with activation of the coagulation or fibrinolytic systems. Br J Haematol, 1985. 60(4): p. 715-22.
Karlsson, R., SPR for molecular interaction analysis: a review of emerging application areas. J Mol Recognit, 2004. 17(3): p. 151-61.
Kourlas, H. and D. S. Schiller, Pegaptanib sodium for the treatment of neovascular age-related macular degeneration: a review. Clin Ther, 2006. 28(1): p. 36-44.
Ku, T. H., et al., Nucleic Acid Aptamers: An Emerging Tool for Biotechnology and Biomedical Sensing. Sensors (Basel), 2015. 15(7): p. 16281-313.
Lane, D. A., et al., Characterization of serum fibrinogen and fibrin fragments produced during disseminated intravascular coagulation. Br J Haematol, 1978. 40(4): p. 609-15.
Maarten T. M. Raijmakers, P. C. H. F. M. H. L. V., PhD Fedde van der Graaf, PhD, Collection of Blood Specimens by Venipuncture for Plasma-Based Coagulation Assays: Necessity of a Discard Tube. American Journal of Clinical Pathology, 2010. 133(2): p. 331-335.
Medcalf, R. L., What drives “fibrinolysis”? Hamostaseologie, 2015. 35(4): p. 303-10.
Nolan, T. E., R. P. Smith, and L. D. Devoe, Maternal plasma D-dimer levels in normal and complicated pregnancies. Obstet Gynecol, 1993. 81(2): p. 235-8.
Nomura, H., et al., Negative predictive value of D-dimer for diagnosis of venous thromboembolism. Int J Hematol, 2008. 87(3): p. 250-5.
Oliphant, A. R., C. J. Brandt, and K. Struhl, Defining the sequence specificity of DNA-binding proteins by selecting binding sites from random-sequence oligonucleotides: analysis of yeast GCN4 protein. Mol Cell Biol, 1989. 9(7): p. 2944-9.
Riley, R. S., et al., Widely Used Types and Clinical Applications of D-Dimer Assay. Laboratory Medicine, 2016. 47(2): p. 90-102.
Roy T. Smith, M. D. C.-H. T. a., Ph.D., Fibrin Degradation Products in the Postoperative Period: Evaluation of a New Latex Agglutination Method. American Journal of Clinical Pathology, 1 Nov. 1973. 60(5): p. 644-647.
Ruzicka, J., A. D. Carroll, and I. Landesmaki, Immobilization of proteins on agarose beads, monitored in real time by bead injection spectroscopy. Analyst, 2006. 131(7): p. 799-808.
Selvaraj, J. N., et al., Recent mycotoxin survey data and advanced mycotoxin detection techniques reported from China: a review. Food Addit Contam Part A Chem Anal Control Expo Risk Assess, 2015. 32(4): p. 440-52.
Siddiqui, M. Z., Monoclonal antibodies as diagnostics; an appraisal. Indian J Pharm Sci, 2010. 72(1): p. 12-7.
Tuerk, C. and L. Gold, Systematic evolution of ligands by exponential enrichment: RNA ligands to bacteriophage T4 DNA polymerase. Science, 1990. 249(4968): p. 505-10.
Walker, J. B. and M. E. Nesheim, The molecular weights, mass distribution, chain composition, and structure of soluble fibrin degradation products released from a fibrin clot perfused with plasmin. J Biol Chem, 1999. 274(8): p. 5201-12.
Walter G. Guder, S. N., Hermann Wisser, Bernd Zawta, Diagnostic Samples: From the Patient to the Laboratory: The Impact of Preanalytical Variables on the Quality of Laboratory Results, 4th, Updated Edition. Wiley-Blackwell, September 2009.
Zhou, J. and J. Rossi, Aptamers as targeted therapeutics: current potential and challenges. Nat Rev Drug Discov, 2017. 16(3): p. 181-202.

### E. Sequences

>2Q9I: A|PDBID|CHAIN|SEQUENCE
SEQ ID NO: 38
VSEDLRSRIEVLKRKVIEKVQHIQLLQKNVRAQLVDMKRLEVDIDIKIR
SCRGSCSRALAREVDLKDYEDQQKQLEQVIAKDLLPSR
>2Q9I: B|PDBID|CHAIN|SEQUENCE
SEQ ID NO: 39
DNENVVNEYSSELEKHQLYIDETVNSNIPTNLRVLRSILENLRSKIQKL
ESDVSAQMEYCRTPCTVSCNIPVVSGKECEEIIRKGGETSEMYLIQPDS
SVKPYRVYCDMNTENGGWTVIQNRQDGSVDFGRKWDPYKQGFGNVATNT
DGKNYCGLPGEYWLGNDKISQLTRMGPTELLIEMEDWKGDKVKAHYGGF
TVQNEANKYQISVNKYRGTAGNALMDGASQLMGENRTMTIHNGMFFSTY
DRDNDGWLTSDPRKQCSKEDGGGWWYNRCHAANPNGRYYWGGQYTWDMA
KHGTDDGVVWMNWKGSWYSMRKMSMKIRPFFPQQ
>2Q9I: C|PDBID|CHAIN|SEQUENCE
SEQ ID NO: 40
KMLEEIMKYEASILTHDSSIRYLQEIYNSNNQKIVNLKEKVAQLEAQCQ
EPCKDTVQIHDITGKDCQDIANKGAKQSGLYFIKPLKANQQFLVYCEID
GSGNGWTVFQKRLDGSVDFKKNWIQYKEGFGHLSPTGTTEFWLGNEKIH
LISTQSAIPYALRVELEDWNGRTSTADYAMFKVGPEADKYRLTYAYFAG
GDAGDAFDGFDFGDDPSDKFFTSHNGMQFSTWDNDNDKFEGNCAEQDGS
GWWMNKCHAGHLNGVYYQGGTYSKASTPNGYDNGIIWATWKTRWYSMKK
TTMKIIPFNRLTIGEGQQHHLGGAKQAGDV
2Q9I: M|PDBID|CHAIN|SEQUENCE
SEQ ID NO: 41
MHRPY
>2Q9I: S|PDBID|CHAIN|SEQUENCE
SEQ ID NO: 42
GHRP

1. An isolated nucleic acid encoding the amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, or SEQ ID NO: 30, or any fragment or variant thereof comprising at least 84% sequence identity thereto.

2. The isolated nucleic acid of claim 1, wherein the nucleic acid comprises sequence as set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, or any fragment or variant thereof comprising at least 87% sequence identity thereto

3. The isolated nucleic acid of claim 2, wherein the nucleic acid sequence is a truncation of SEQ ID NO: 1.

4. The isolated nucleic acid of claim 3, wherein the truncated nucleic acid sequence comprises SEQ ID NO: 16, SEQ ID NO: 17, SEQ ID NO: 18, SEQ ID NO: 19, or SEQ ID NO: 20.

5. The RNA equivalent of any of the nuclei acids of claim 1.

6. The isolated nucleic acid of claim 1, further comprising a detectable tag.

7. The isolated nucleic acid of claim 6, wherein the detectable tag comprises a latex bead, magnetic bead, fluorescence label; fluorescent probe, chemiluminescent labels, radiolabels, and/or nanoparticle probe.

8. A composition comprising one or more of the isolated nucleic acids of claim 1.

9. A kit comprising one or more of the nucleic acids of claim 1.

10. A method of detecting D-dimer in a subject comprising obtaining a biologic sample from the subject and measuring the concentration of D-dimer in the subject using one or more of the aptamers of claim 1.

11. A method of detecting deep venous thrombosis (DVT), pulmonary embolism (PE), or disseminated intravascular coagulation (DIC) comprising obtaining a biologic sample from the subject and measuring the concentration of D-dimer in the subject using one or more of the aptamers of claim 1; wherein a concentration of D-dimer above 500 ng/mL indicates that the subject has DVT, PE, or DIC.

12. The method of claim 11, wherein the concentration of D-dimer indicates the subject has DVT, PE, or DIC, said method further comprises administering to the subject an anticoagulant.

13. A method of treating deep venous thrombosis (DVT), pulmonary embolism (PE), or disseminated intravascular coagulation (DIC) comprising administering to a subject an anticoagulant conjugated to one or more of the aptamers of claim 1.
